(19) United States
(12) Patent Application Publication
(10) Pub. No.: US 2021/0330455 A1
(43) Pub. Date: Oct. 28, 2021

(54) DEVICES, SYSTEMS, AND METHODS FOR A COLLAPSIBLE REPLACEMENT HEART VALVE

(71) Applicant: ReValve Solutions Inc., Bainbridge Island, WA (US)

(72) Inventors: Julie Logan Sands, McLean, VA (US); Kenneth Eugene Perry, Bainbridge Island, WA (US); Edward Ian Wulfman, Woodinville, WA (US); Phillip Andrew Ahrens, Bainbridge Island, WA (US)

(73) Assignee: ReValve Solutions Inc., Bainbridge Island, WA (US)

(21) Appl. No.: 17/240,914

(22) Filed: Apr. 26, 2021

Related U.S. Application Data
(60) Provisional application No. 63/015,353, filed on Apr. 24, 2020.
(60) Provisional application No. 63/025,881, filed on May 15, 2020.

(51) Int. Cl. A61F 2/24

(52) U.S. Cl. CPC A61F 2/2418; A61F 2220/0075; A61F 2/2409

(57) ABSTRACT

Disclosed herein are systems and methods for a replaceable heart-valve system comprising a tubular braided frame, wherein the tubular braided frame comprises an inflow end, an outflow end, and comprising one or more commissure posts extending vertically away from the inflow end. The valve system further comprises a leaflet assembly, wherein the leaflet assembly comprises at least one valve leaflet, wherein the at least one leaflet comprises an inflow end, an outflow end, and one or more commissure tabs extending horizontally away from the inflow end; and wherein the one or more commissure tabs of the at least one leaflet is connected to the one or more commissure posts of the tubular braided frame.

### CROSS-REFERENCE TO RELATED APPLICATIONS

This application claims benefit to U.S. Provisional Application No. 63/015,353, filed on Apr. 24, 2020, titled Devices, Systems and Methods Relating To Quickly Implantable and Replaceable Heart Valves; and claims benefit to U.S. Provisional Application No. 63/025,881, filed on May 15, 2020, titled Devices, Systems, and Methods For a Collapsible and Expandable Replacement Heart Valve; the contents of all of which are incorporated herein by this reference as though set forth in their entirety, and to which priority and benefit are claimed.

### FIELD OF USE

The present disclosure relates generally to replacement heart-valve technology, and more specifically to devices, systems, and methods for a collapsible and expandable heart-valve assembly that is highly flexible, resilient, retractable, and replaceable.

### BACKGROUND

Heart-valve intervention, such as full open-heart surgery, is often required to treat diseases of one or more of the four heart valves, which work together to keep blood properly flowing through the heart. Replacement and/or repair of a heart valve is often required when a valve is “leaky” (e.g., there is mitral valve regurgitation) or when a valve is narrowed and does not open properly (e.g., mitral valve stenosis). Typically, heart-valve replacement, such as mitral-valve replacement, involves replacement of the heart's original (native) valve with a replacement, mechanical and/or tissue (bioprosthetic) valve.

Common problems with the replacement of valves and/or the frames carrying them comprise: degradation of the leaflets (valve-like structure); breaking or failing frames, particularly with laser-cut nitinol frames; and undesirable changing in size of the native valve annulus. Replacement heart valves pose additional problems after they are implanted. For example, the replacement valve may move or migrate after it is placed in a desired location in the heart, or its location may not permit proper directional flow of blood during delivery. Replacement valves are also not readily retrievable, most often because such removal can damage the surrounding heart tissue. This can be particularly problematic, for example, if the replacement valve is not properly and accurately placed into position when it is implanted in the native heart, as well as when the replacement valve starts failing, which may occur years after initial implantation. An additional problem is that typical replacement valves, especially laser-cut valve frames, are relatively stiff and inflexible, resulting in a valve that does not flex with the dynamic movements of the pumping heart. Such inflexible valves do not conform to such dynamic movements, which can cause trauma to the heart surfaces, cause breaks in the frame itself, otherwise cause or exacerbate problems during or after implantation.

Thus, what is needed are devices, systems, and methods for a replacement heart valve that enables compact and secure delivery into the desired location within the heart and convenient control of expansion and retraction of the heart valve when being implanted or removed, such as via a catheter. Such a replacement heart valve should provide adequate flexibility to match, conform to, or otherwise not impede the heart's dynamic movement. And it should ensure proper directional blood flow through the heart during and after the replacement procedure.

### SUMMARY OF THE DISCLOSURE

The following presents a simplified overview of the example embodiments in order to provide a basic understanding of some embodiments of the present disclosure. This overview is not an extensive overview of the example embodiments. It is intended to neither identify key or critical elements of the example embodiments nor delineate the scope of the appended claims. Its sole purpose is to present some concepts of the example embodiments in a simplified form as a prelude to the more detailed description that is presented herein below. It is to be understood that both the following general description and the following detailed description are exemplary and explanatory only and are not restrictive.

The present disclosure is directed to devices, systems, and method for a collapsible, replacement heart-valve assembly that is highly flexible, resilient, retractable, and replaceable. As disclosed herein, the valve assembly has the capability to be replaced years after implantation if problems, such as recurrent mitral valve regurgitation, arise.

In a preferred embodiment, a heart-valve system may comprise a tubular braided frame, wherein the tubular braided frame comprises an inflow end, an outflow end. The valve system may further comprise a leaflet assembly, wherein the leaflet assembly comprises at least one valve leaflet, wherein the at least one leaflet comprises an inflow end, an outflow end, and one or more commissure tabs extending horizontally away from the inflow end; and wherein the one or more commissure tabs of the at least one leaflet is connected to the one or more commissure posts of the tubular braided frame. In some embodiments, the tubular braided frame may comprise one or more commissure posts extending vertically away from the inflow end.

Still other advantages, embodiments, and features of the subject disclosure will become readily apparent to those of ordinary skill in the art from the following description wherein there is shown and described a preferred embodiment of the present disclosure, simply by way of illustration of one of the best modes best suited to carry out the subject disclosure. As will be realized, the present disclosure is capable of other different embodiments and its several details are capable of modifications in various obvious embodiments all without departing from, or limiting, the scope herein. Accordingly, the drawings and descriptions will be regarded as illustrative in nature and not as restrictive.

### BRIEF DESCRIPTION OF THE DRAWINGS

The accompanying drawings, which are incorporated in and constitute a part of this specification, illustrate embodiments of the disclosure and together with the general description of the disclosure given above and the detailed description of the drawings given below, serve to explain the principles of the disclosure. In certain instances, details that are not necessary for an understanding of the disclosure or that render other details difficult to perceive may have been omitted.

FIG. 1 generally illustrates an embodiment of a collapsible, heart-valve assembly system as disclosed herein.

FIG. 2 generally illustrates an embodiment of a tubular, braided retrievable frame as disclosed herein.

FIG. 3 generally illustrates an embodiment of a tubular, braided retrievable frame as disclosed herein.

FIG. 4 generally illustrates an embodiment of a tubular, braided retrievable frame as disclosed herein.

FIG. 5 generally illustrates an embodiment of a tubular, braided retrievable frame as disclosed herein.

FIG. 6 generally illustrates an embodiment of a tubular, braided retrievable frame as disclosed herein.

FIG. 7 generally illustrates an embodiment of a leaflet panel as disclosed herein.

FIG. 8 generally illustrates an embodiment of a leaflet panel as disclosed herein.

FIG. 9 generally illustrates an embodiment of a Z-valve insert as disclosed herein.

FIGS. 10A-10C generally illustrate embodiments leaflet-panel patterns as disclosed herein.

FIGS. 11A-10D generally illustrate embodiments leaflet-panel patterns as disclosed herein.

FIG. 12 generally illustrates an embodiment of a collapsible, heart-valve assembly system as disclosed herein.

FIG. 13 generally illustrates an embodiment of a collapsible, heart-valve assembly system as disclosed herein.

FIG. 14 generally illustrates an embodiment of a collapsible, heart-valve assembly system as disclosed herein.

FIG. 15 generally illustrates an embodiment of a collapsible, heart-valve assembly system as disclosed herein.

FIG. 16 generally illustrates an embodiment of a collapsible, heart-valve assembly system as disclosed herein.

FIG. 17 generally illustrates an embodiment of a collapsible, heart-valve assembly system as disclosed herein.

FIG. 18 generally illustrates an embodiment of a collapsible, heart-valve assembly system as disclosed herein.

FIG. 19 generally illustrates an embodiment of a retrieval system of a collapsible, heart-valve assembly as disclosed herein

FIG. 20 generally illustrates an embodiment of a collapsible, heart-valve assembly system as disclosed herein.

FIG. 21 generally illustrates an embodiment of a deployment system for a collapsible, heart-valve assembly system as disclosed herein.

FIG. 22 generally illustrates an embodiment of a deployment system for a collapsible, heart-valve assembly system as disclosed herein.

FIG. 23 generally illustrates an embodiment of a deployment system for a collapsible, heart-valve assembly system as disclosed herein.

FIG. 24 generally illustrates an embodiment of a deployment system for a collapsible, heart-valve assembly system as disclosed herein.

FIG. 25 generally illustrates an embodiment of a deployment system for a collapsible, heart-valve assembly system as disclosed herein.

FIG. 26 generally illustrates an embodiment of a deployment system for a collapsible, heart-valve assembly system as disclosed herein.

FIG. 27 generally illustrates an embodiment of a deployment system for a collapsible, heart-valve assembly system as disclosed herein.

FIG. 28 generally illustrates an embodiment of a deployment system for a collapsible, heart-valve assembly system as disclosed herein.

FIG. 29 generally illustrates an embodiment of a corset of a deployment system for a collapsible, heart-valve assembly system as disclosed herein.

FIG. 30 generally illustrates an embodiment of a deployment system for a collapsible, heart-valve assembly system as disclosed herein.

FIG. 31 generally illustrates an embodiment of a deployment system for a collapsible, heart-valve assembly system as disclosed herein.

FIG. 32 generally illustrates an embodiment of a collapsible, heart-valve assembly system as disclosed herein.

FIG. 33 generally illustrates an embodiment of a deployment system for a collapsible, heart-valve assembly system as disclosed herein.

FIG. 34 generally illustrates an embodiment of a braiding configuration for the retrievable frame as disclosed herein.

FIG. 35 generally illustrates an embodiment of a braiding configuration for the retrievable frame as disclosed herein.

FIG. 36 generally illustrates an embodiment of a braiding configuration for the retrievable frame as disclosed herein.

FIG. 37 generally illustrates an embodiment of a braiding configuration for the retrievable frame as disclosed herein.

FIG. 38 generally illustrates an embodiment of a braiding configuration for the retrievable frame as disclosed herein.

FIG. 39 generally illustrates an embodiment of a braiding configuration for the retrievable frame as disclosed herein.

FIG. 40 generally illustrates an embodiment of a braiding configuration for the retrievable frame as disclosed herein.

### DETAILED DESCRIPTION OF EMBODIMENTS

Before the present systems and methods are disclosed and described, it is to be understood that the systems and methods are not limited to specific methods, specific components, or to particular implementations. It is also to be understood that the terminology used herein is for the purpose of describing particular embodiments only and is not intended to be limiting. Various embodiments are described with reference to the drawings. In the following description, for purposes of explanation, numerous specific details are set forth in order to provide a thorough understanding of one or more embodiments. It may be evident, however, that the various embodiments may be practiced without these specific details. In other instances, well-known structures and devices are shown in block diagram form to facilitate describing these embodiments.

Disclosed herein is a collapsible heart-valve assembly system comprising at least a braided, collapsible frame and leaflet assembly that together serve to provide a sealing portion. The valve assembly may be delivered through a catheter and may perform as either a standalone valve replacement or placed within an existing receiver structure. The valve assembly may further comprise attachments and additional features for catheter delivery, positioning, partial deployment, and retrieval.

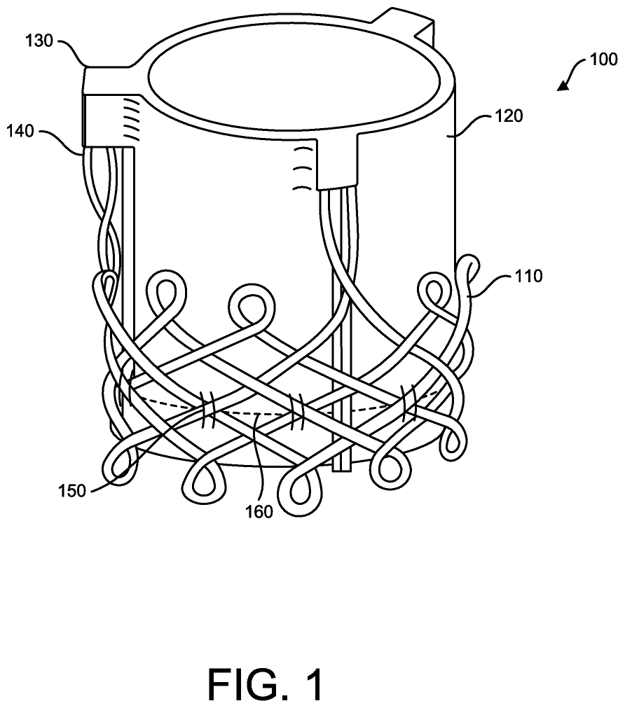

FIG. 1 generally illustrates an embodiment of a collapsible, heart-valve assembly system as disclosed herein. As shown in FIG. 1, a valve assembly 100 may comprise a tubular, braided frame 110 and a leaflet assembly 120 incorporated into the frame 110, with the frame 110 comprising one or more commissure posts 140. Commissure posts 140 may be defined as attachment points for the downstream portion of the leaflet assembly 120. In a preferred embodiment, a frame comprises three commissure posts, though it is common for a frame to have two or four commissure posts; and it may have more than four or none at all. Commissure posts 140 do not need to be symmetrically disposed around the valve, nor must they be the same height. When compressed, the commissure posts can elastically deform to accommodate not deforming a non-flexible leaflet material.

The valve assembly 100 may further comprise one or more tabs 130, wherein the tabs 130 are sewn to the commissure posts 140. The valve assembly 100 may also comprise a base stitch 150 along a row of lashed crossing points of the frame 110, wherein the base stitch 150 connects the frame 110 to the leaflet assembly 120 along the circumference of the leaflet assembly's centerline 160. A base stitch 150 may be defined as the stitching line that delineates the inflow edge of a functional valve assembly 100.

Thus, as shown in FIG. 1, a valve system may comprise a tubular braided frame, wherein the tubular braided frame comprises an inflow end, an outflow end, and one or more commissure posts extending vertically away from the inflow end. The valve system may further comprise a leaflet assembly, wherein the leaflet assembly comprises at least one valve leaflet, wherein the at least one leaflet comprises an inflow end, an outflow end, and one or more commissure tabs extending horizontally away from the inflow end; and wherein the one or more commissure tabs of the at least one leaflet is connected to the one or more commissure posts of the tubular braided frame.

The valve assembly disclosed herein is novel and an improvement over the prior art because it combines a minimal braided, wire structure with a novel leaflet-assembly design, wherein the integration of both together in a strategic manner provides a valve that may be compressed to a very low profile that is much smaller than other existing percutaneously delivered valves. Additionally, the design of the valve assembly creates an option for ease of removal, either by percutaneous techniques or by minimally invasive surgical techniques.

Benefits of this disclosure over prior art include but are not limited to a less-invasive and less-traumatic puncture to accommodate delivery of the valve assembly. Further, the profile of the valve assembly and the strategic combination of wire-braid frame, leaflet assembly, and attachment strategy allows for a more flexible delivery system. This is due to the nature of the flexible wire frame and because the valve is relatively short and allows for flexibility in the delivery catheter. The combination of these factors allows for less traumatic delivery, more precise delivery, and a greater number of options on how to deliver.

Additionally, the ability to remove by percutaneous or minimally invasive techniques is a unique advantage of this valve assembly. Current state-of-the-art implanted valves, if they become malfunctioning or disabled, must either be removed by major surgical techniques or by implanting a second valve inside the malfunctioning valve. Both of these options have major drawbacks. Major surgery often is obviated by a patient because of age or physical condition. And implanting a valve inside an existing valve compromises the newly implanted valve and reduces the options if it does not work as intended.

Thus, the present systems, devices, and methods disclosed herein include replacement heart valve frames that are highly flexible, resilient, retractable, and replaceable—including replacement years after implantation if problems such as recurrent valve regurgitation arise.

In one embodiment, the replacement heart valve system comprises: a retrievable frame portion holding a replacement heart valve (i.e., a blood flow regulator such as an array of valve leaflets, a porcine valve leaflets, or other structure that selectively allows/prevents flow of blood through the valve, typically unidirectional); and a receiver frame portion sized and configured to removably retain the retrievable frame portion. Both of the frame portions can advantageously be made of braided nitinol, although other materials can also be used such as stainless steel, cobalt chrome, polymers such as nylon, or laser-cut nitinol. The tissue leaflets, or other blood flow regulator, are constructed to perform the function of a valve to regulate flow through any one of the four orifices in the human heart, including for example the mitral valve, although the system can also be configured for and used/located in the tricuspid valve, the pulmonary valve, or the aortic valve.

The frame portions of the replacement heart valve systems herein, particularly the retrievable frame portion, can be made of a single (or multiple) wire braid. Such frame portions can be constructed, for example, using nitinol wire on a cylindrical mandrel, following an over-under configuration of wire wraps. Where the frames are made of nitinol or other shape-memory material, the frames can be shape-set to produce a stress-free structure after it is expanded in situ. Some advantages of wire-braid frames, for certain situations, is that such wire-braid frames can be constructed such that they are substantially stress-free both when deployed as well as when they are contained and constrained within a delivery or retrieval catheter; except for apical vertices of the frame located at the inflow and outflow ends of the frame, which are bent inwardly due to such in-catheter disposition.

The cardiac valve retrievable frame houses valve leaflets or other unidirectional valve structure. Leaflets can be cut from bovine pericardium (or other materials) and sewn, fused or otherwise attached together along edges and attached to the frame along the inflow edge of the structure.

The replacement heart valve system including each and both of the retrievable frame portion and receiver frame portion, has an inflow end and an outflow end. Inflow references the end of the frame/system that blood flows into the frame/system, while outflow references the end of the frame/system that blood flows out of the frame/system. For example, when the frame/system is deployed in the mitral valve, the outflow end is located in the left ventricle while the inflow end is located in the left atrium.

The frame portions of the heart valve system can be provided separately or as a unit. For example, the retrievable frame portion and receiver frame portion can be surgically implanted or carried in a catheter—either linked together or separately in a single catheter, or separately in separate catheters. The retrievable frame portion and receiver frame portion, both separately and particularly when provided in a single catheter, can be compressed to catheter dimensions as desired, for example as small as 26F, 24F, 18F, 16F, 14F or less. The replacement heart valve system typically provides one-way flow control.

During deployment of the replacement heart valve system, one or both ends of the system is configured to retain mechanical connection with the catheter delivery system such that the replacement heart valve system can be positioned and repositioned through physical motions such as rotation and translation, or a combination thereof. Once fully released from the delivery catheter, the replacement heart valve system secures the receiver frame portion of the system to the target native heart, for example via expansion and/or self-expansion to create a radial force against tissue at the target site, anchoring elements that connect to the target tissue, or otherwise as desired. Such securement contact typically includes contact with the target native heart valve, such as the native heart valve leaflets and annulus, and/or native heart structures on either the inflow and/or outflow sides of the native heart valve. In an embodiment, the receiver frame portion and/or the retrievable frame portion have a cross-section that is generally cylindrical or D-shaped to fill the target orifice. The retrievable frame portion and receiver frame portion may have compatible or identical cross-sectional shapes so that the retrievable frame portion nests within the receiver frame portion.

In some embodiments, the retrievable frame portion and the receiver frame portion are cooperatively sized and configured together to fit together within a single heart catheter for delivery to a target mitral valve or other target location. The retrievable frame portion and the receiver frame portion can be carried in the delivery catheter as a single unit, i.e., in an as-connected form where the two portions are mechanically linked together. This configuration can advantageously allow the delivery catheter to control both portions of the frame system by controlling only one of the frames, for example using the proximal frame portion to control the distal frame portion (the distal frame portion being the frame portion that first exits the end of the delivery catheter).

The retrievable frame portion and the receiver frame portion can also be carried in the delivery catheter in an unconnected form where the two portions are not mechanically linked together. This configuration can advantageously allow the delivery catheter to independently control each of the frame portions and can also increase the flexibility and torsion characteristics of the delivery catheter containing the frames, which can be advantageous both while conveying the delivery catheter to through the patient's body, the vasculature, the desired target, and while delivering the replacement valve at/to the target.

In some embodiments, the retrievable valve frame portion and the receiver frame portion can each be less than about 50 mm, 40 mm, 30 mm, or 25 mm in length.

In certain embodiments, the frame portions can bend to a radius of curvature as low as about 1″, ¾″, ½″, ⅜″, or ¼″ (inches).

In some methods of deployment, the two frame portions can be carried in unconnected format, then mechanically connected to each other while at least a substantial portion of one of the frames (typically the distal frame) has been pushed or otherwise thrust into the target heart, including into the target location within the heart.

These methods can take advantage of the benefits of separated-frame carriage within the delivery catheter while also taking advantage of the ability to control both (or more, if more than two) frame portions via only a single-frame connection with the delivery catheter's control elements. This can also advantageously allow partial deployment then manipulation (even full withdrawal) of the frame system to deploy the receiver frame portion in a better location in the target heart location, or even full deployment then manipulation/withdrawal of the frame system to a more desired location, orientation, etc., at the target location.

After full deployment of the replacement heart valve system in a target location, the retrievable frame portion is removably retained within the receiver frame portion such that removal of the retrievable frame portion from the receiver frame portion comprises gathering and pulling the retrievable frame portion from the receiver frame portion, twisting and pulling the retrievable frame portion from the receiver frame portion, twisting and pushing the retrievable frame portion out of the receiver frame portion, or otherwise as desired. The retrievable frame portion is typically withdrawn from the receiver frame portion without substantially disturbing or disrupting the connection of the receiver frame portion to the target location in the heart, and without harming the native heart tissue. For example, removal of the retrievable frame from the receiver frame does not dislodge replacement valve tissue, target tissue, nor frame elements during such removal as well as during re-insertion of a new valve and retrievable frame assembly. Exemplary forces to remove the retrievable frame from the receiver frame include less than 1.5 pounds, less than 1 pound, less than 0.75 pounds, less than 0.5 pounds or less than 0.25 pounds.

The retrievable frame portion retained within the receiver frame portion can have a high degree of flexibility. In certain embodiments, the retrievable frame portion is substantially more flexible than the receiver frame portion. The retrievable frame portion and/or receiver frame portion can be made of braided wire. The braided wire can be nitinol or stainless steel or other material as desired.

In some embodiments, the retrievable frame portion and the receiver frame portion can be cooperatively sized and configured to a) removably retain the retrievable frame portion in the receiver frame portion against blood flow forces in a heart valve holding the replacement heart valve system and b) release the retrievable frame portion from the receiver frame portion without harming surrounding target heart tissue. The heart valve can be a mitral valve or other target heart valve. The native heart tissue can be native heart valve tissue.

The retrievable frame portion and the receiver frame portion can be cooperatively sized and linked to fit within a single heart catheter for delivery to a target mitral valve. The retrievable frame portion can be retained within the receiver frame portion such that removal of the receiver frame portion from the receiver frame portion can comprise pushing or pulling the retrievable frame portion through the receiver frame portion towards an inflow end of the receiver frame portion. The retrievable frame portion can be retained within the receiver frame portion such that removal of the receiver frame portion from the receiver frame portion can comprise pushing or pulling the retrievable frame portion from the receiver frame portion towards an outflow end of the receiver frame portion. The retrievable frame portion can be configured such that the retrievable frame portion substantially does not touch a side of a native heart chamber when the retrievable frame portion is placed in the native heart chamber.

Intersections of the braided wire comprise crossing angles of 20° to 30° in a fully expanded configuration or crossing angles of about 0° to 5° in a fully compressed configuration such that compressed wires are substantially parallel to each other. Crossing angles within the retrievable frame portion or the receiver frame portion can be uniform or non-uniform.

Apical angles of the braided wire are 20° to 30° in a fully expanded configuration, or 160° to 180° in a fully compressed configuration such that the wire leading into and out of the apex is substantially parallel to itself. Apical angles within the retrievable frame portion or the receiver frame portion can be uniform or non-uniform.

A combination of the retrievable frame portion within the receiver frame portion together has a flexibility such that the combination can bend to a radius of curvature less than about 1″, ¾″, ½″, ⅜″, or ¼.″

Thus, methods can comprise: During the implanting, removing the retrievable frame portion from the receiver frame portion and re-inserting retrievable frame portion into the receiver frame portion. The methods can also comprise: After the implanting is completed, accessing only via catheter the replacement heart valve system as located in the target heart valve and removing the valve and retrievable frame assembly from the receiver frame portion. Such methods can further comprise: After removing the valve and retrievable frame assembly from the receiver frame portion, replacing the valve and retrievable frame assembly in the receiver frame portion with a second, different valve and retrievable frame assembly.

These embodiments enable facilitation of the replacement of a heart blood flow regulator, such as a tissue valve, after initial placement, without excessive harm to any of the patient's body, heart or target heart valve. This is highly advantageous because replacement valves often suffer problems that require replacement of the heart valve. Common problems with replacement valves and/or the frames carrying them include the leaflets can degrade, the frames can break or otherwise fail, particularly with laser-cut nitinol frames, and the native valve annulus can undesirably change in size. The current systems, etc., reduce or even eliminate one or more of these problems.

In some embodiments, commissure posts can be provided on a “free end” of the retrievable frame portion, which means the end of the retrievable frame portion that is not connected to the receiver frame portion. The commissure posts can be configured to anchor the leaflet tissue, for example three commissure posts can be disposed at substantially equidistant locations around the receiver frame portion to provide connection locations for each commissure of a three-leaflet valve. If the commissures of the three-leaflet valve are not equidistant, then the commissure posts of the valve frame can be configured to match the locations of such valve commissures. In another example, two commissure posts can be used for a bi-leaflet valve. Other valve-commissure post orientations and configurations can also be provided, including that the number of commissure posts does not have to equate to the number of leaflets. Such commissure posts can provide anchoring points to hold the leaflets or other unidirectional valve structure and can also provide deployment attachment points for the delivery and/or retrieval catheter.

Attachment of the leaflets can also be made to a cuff carried by the retrievable frame, or other materials can be provided to create a sealing zone at the inflow end to prevent the flow of blood between the retrievable frame structure and the receiver frame, which can be cylindrical. Gripping, positioning, securement, and retrieval elements such as grippers, positioners, anchors, or retrieval hooks can be included in the retrievable frame portion and receiver frame portions. Such securement elements permit a physician or other user to accurately place the structure in a target heart valve and to easily retrieve the retrievable frame structure post-deployment if desired. Exemplary methods to implement such easy, low-impact removal include known heart procedures such percutaneous access, for example via catheter under fluoroscopy.

In some embodiments, the retrievable frame portion, for example when made of braided wire, can be configured to be inserted into a primary receiver frame portion that is the initial frame of the replacement heart valve system to be implanted into the target site; this receiver frame portion may be permanently implanted. The retrievable frame valve assembly is then implanted or inserted as a secondary portion of the replacement heart valve system; this retrievable frame portion/valve and retrievable frame assembly may not be permanently implanted but instead can be easily withdrawn and replaced, in some embodiments without harm to the target native heart tissue. This receiver frame portion is typically configured to be permanently implanted into the native target site, while the valve assembly (i.e., an assembly comprising the blood flow regulator valve and the retrievable frame portion) is configured to be removable, for example by a catheter procedure, for up to several or more years after implant. The system of the braided wire retrievable frame portion and receiver frame portion can thus be configured so that they work in concert to connect to the cardiac tissue, hold tissue valve leaflets in place to control blood flow, and be strong enough to resist the forces of cardiac blood flow, yet also allow replacement of the replacement tissue valve and/or the valve and retrievable frame assembly easily and without damaging the native heart.

This division into separated entities can also allow the valve frame, i.e., the retrievable frame portion, to be constructed with a lighter wire than if the full valve system were one piece. Exemplary embodiments of such lighter wire include nitinol wire from 0.009″ to 0.015″ diameter. One advantage of such systems is that the lighter secondary/retrievable valve portion can be compressed more than a stiffer frame and thus the secondary/retrievable valve portion can be delivered in and retrieved by a smaller catheter. This also allows a greater range of patients to receive the treatment.

Another advantage of the current braided wire frames is that, when compressed to catheter dimensions, the retrievable frame portion experiences significant strain values, including peak strain values, only in the frame apices at either end of the retrievable frame. In contrast, typical percutaneous replacement valves with rows of zig-zag features experience large outward strains along the full length of the structure. Thus, the current braided wire frames allow for a smaller bend radius of curvature (less than 0.75″, 0.5″, etc.), less force and torque required for positioning/repositioning the retrievable valve frame, and the ability to “stack” several structures (e.g. a receiver/anchor system along with the valve structure) into a single catheter for a rapid sequential deployment. Another advantage of the current braided wire frames is greater positional accuracy and ease of repositioning, for example because of the reduced outward force profile of the frames herein.

The wire(s) of the braid can move or slide independently of each other at each crossing. This slippage allows for a high degree of design and configuration options so that a user can configure a replacement heart valve system, including individual portions and parts thereof, for specific situations such as specific patients or target sites. The strength of the braided frame can be selected by providing a specific, desired number of wraps and/or configurations of the wire(s). Exemplary outward radial forces exerted by the frame portions herein when deployed at a target location include at least about 0.15 N, 0.2 N, 03 N, and 0.4 N. Exemplary outward radial forces exerted by the frame portions herein when deployed at a target location include about 1 N, 1.5 N, and 2 N.

If desired, the wraps can be braided in a zig zag pattern and braid angle. Examples of such braid angles where the braided wires cross each other include crossing angles of 15°, 20°, 30° or 45° in the as-deployed configuration. Examples of such braid angles where the braided wires cross each other include about 0° to 5° in the compressed (in-catheter) configuration such that the crossing wires are substantially parallel to each other. Such braid-crossing angles can be uniform throughout the frame or can be non-uniform for example to implement differential braid/frame resiliencies or radial pressures in different locations within a frame.

A radial force is created by each bend, or “elbow,” in the apexes in the wire structure. Thus, the frames can be precisely configured for specifically desired forces such as radial forces that help keep the valve and retrievable frame assembly expanded properly and housed in the receiver frame, or to allow proper support of leaflet function, by selecting a desired number of wraps. Forces exerted by the wire braid can also be configured by selecting or modifying a combination of parameters such as wire diameter, “elbow” angles, number of wire crossings, and zig zags around the circumference of the frame. This allows for selected configuration of the frames for specific performance criteria such as valve closing forces, delivery forces, deployment forces, and retrieval forces. Exemplary apice or elbow angles in the compressed form (in-catheter) of the frame include about 160° to 180° such that the wire leading into and out of the apex is substantially parallel to itself. Exemplary apice or elbow angles in the as-deployed form (expanded configuration) of the frame include about 60°, 70°, 80°, to 90°. Such apex angles can be uniform throughout the frame, or can be non-uniform; for example, either different apical angles from one apex to the next, or by having different angles within a single apex. Non-uniform apical angles can help to implement differential braid/frame resiliencies or radial pressures in different locations within a frame.

Releasable gripping and locking elements can be integrated into the frames to provide desire manipulation, control, and anchoring of the retrievable frame to a previously placed receiver.

In some embodiments, the unidirectional blood-flow regulator contains tissue leaflets, which contribute moveable surfaces that are biocompatible, durable, and capable of opening and closing, with a finite region of coaptation to seal off and prevent blood flow. The leaflets can be attached to each other, for example, through sewing with suture material and/or tissue welding. Tissue and fabric materials for the leaflets can be chosen to control tissue ingrowth and endotheliazation, for example to selectively promote or inhibit tissue ingrowth and endotheliazation, so that the retrievable frame portion can be fully removed post-deployment. Frame materials, including combinations of frame materials, can also be chosen and configured to control tissue ingrowth/endotheliazation.

Securement elements such as loops, hooks, sutures and the like, as well as combinations thereof, configured to remove the retrievable frame portion post-deployment are configured so that the retrievable frame portion, typically including the valve carried therein, is drawn down to a smaller size such that the retrievable frame portion can be withdrawn from the receiver frame portion. As one example, the retrievable frame portion can be drawn down to one-half its fully expanded diameter. The retrievable frame portion can then be remotely compressed further if desired, for example down to 20-30% of full diameter. The retrievable frame portion can then be pulled into a catheter or sheath, for instance a 24F sheath, permitting the entire structure to be pulled into a catheter for removal from the target valve site. Such removal can be effected transapically, transseptally, transaortically, or otherwise as desired. Exemplary removal elements include a string or wire that resides in situ in the retrievable frame portion while it is deployed in the target site, which string can be snared be a retrieval catheter and the twisted or otherwise used to draw down or lessen the diameter of the retrievable frame portion so that the retrievable frame can be pulled through or pushed from the receiver frame portion for removal and replacement. This can reduce the overall force used to push/pull the device out of the receiver frame portion and into a removal sheath in the retrieval catheter.

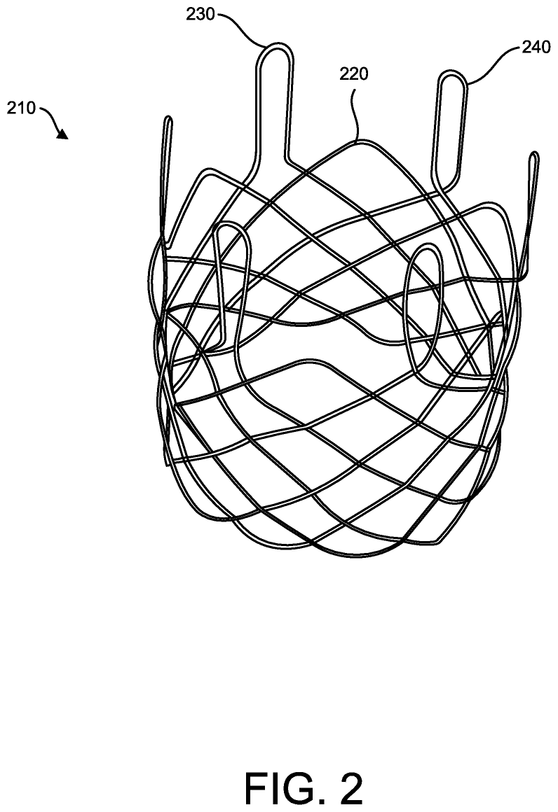

FIG. 2 generally illustrates an embodiment of a tubular, braided retrievable frame as disclosed herein. As shown in FIG. 2, a frame may be configured for implantation into a native valve. The retrievable frame portion 210 is made from wire woven in zig zags that have apices or elbows 220 at the top and bottom, i.e., at the inflow and outflow ends. The wire braid is circumferentially additive, which means that each additive turn of the wire around the circumference adds density to the braid weave/frame and therefore adds to potential radial force. At the inflow end in this example of a mitral valve replacement system—which can also be the distal end relative to a delivery catheter or proximal end relative to a retrieval catheter that pulls on such proximal end to remove the retrievable frame portion 210 from a receiver frame portion—some elbows or apexes/apices can be configured to form commissure post 230 (typically there are three such posts but the number can vary as desired) and some elbows can be configured to form a delivery and or retrieval feature such as catheter attachment loop 240. The frame can vary in size, for example when configured for a mitral valve the frame can be 2-3 cm in diameter and 2-3 cm in length. The braided wire can, for example, be nitinol or stainless steel with a wire diameter from 0.010″ to 0.020″, for example about 0.014″. Thicker or thinner wires can be selected as desired, for example to configure stiffer or less stiff frames.

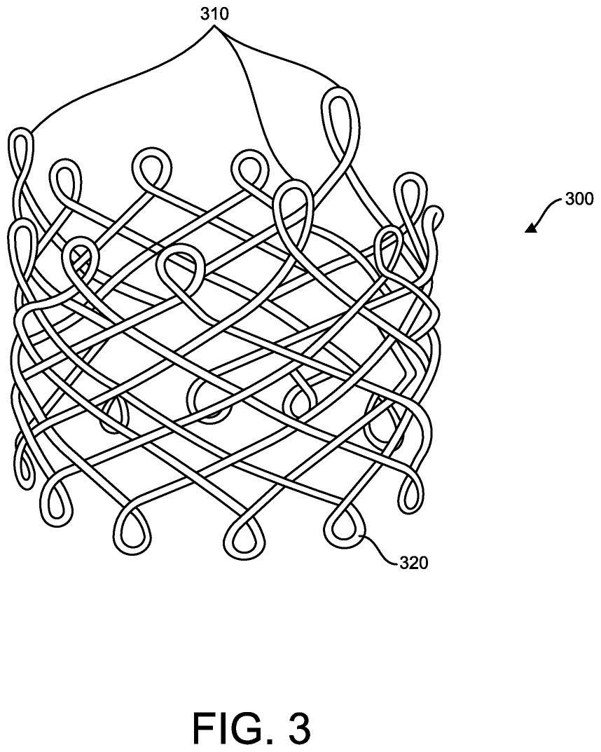

FIG. 3 generally illustrates an embodiment of a tubular, braided retrievable frame as disclosed herein. A braided, valve frame may be defined as a single or multi-wire, braided, self-expanding frame that supports the leaflets and provides a sealing portion. As shown in FIG. 3, the tubular frame 300—with corresponding length, diameter, distal end, and a proximal end—comprises one or more commissure posts 310 at the distal end and loops 320 at both distal and proximal ends. The commissure posts 310 typically extend out from the tubular frame 300 by a minimum amount, for example from 10-30% of the length of the frame 300. The loops 320 may be a simple 300-360-degree loop back of the material making up the frame 300, or may be more than 360 degrees, i.e. two turns of the frame material. The loops 320 provide a stable end to the frame 300, allow for means of looping sutures to a delivery mechanism, provide an attachment means for the leaflet structure, and reduce the peak stresses/strains at the turns. These features can be designed to enhance or reduce the radial force of the system. Additionally, the loops 320 may be used for positioning radiopaque markers for visibility under fluoroscopy.

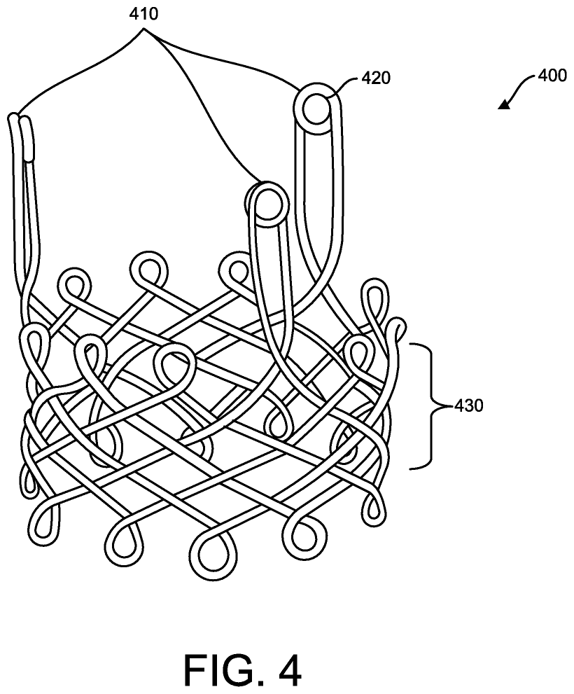

FIG. 4 generally illustrates an embodiment of a tubular, braided retrievable frame as disclosed herein. As shown in FIG. 4, a braided frame 400 may vary in size. For example, the commissure posts 410 may extend further from the distal end of the frame 400. In this embodiment, the commissure posts 410 have a loop 420 that is greater than 360 degrees, creating nearly two full loops. The center 430 of the main body of the frame 400 provides a stable centerline between distal and proximal ends of the frame 400, where a suture line can be made to sew the proximal end of the leaflet structure. An embodiment of this is braid lashing, defined as sutures used to constrain the motion of crossing points in the braid and may be horizontally and/or vertically oriented. The proximal end of the frame 400 may typically be used to provide structure to connect the leaflet structure in a 360-degree suture line and to create a sealing zone for valve function when the valve is inserted into a receiver. A sealing zone generally refers to the region on the outside of the frame between an inflow stitch; an inflow stitch is generally parallel to and on the inflow side of the base stitch and serves to attach a leaflet assembly to a frame or cuff and base stitch so as to provide a larger area over which sealing occurs. Sealing, such as through the use of a sealing ring, refers to the prevention of blood flow from either side while the leaflets provide flow control. This centerline is stable because when the braided structure is compressed for delivery, the structure elongates and so the proximal and distal ends move away from each other equidistant from the center.

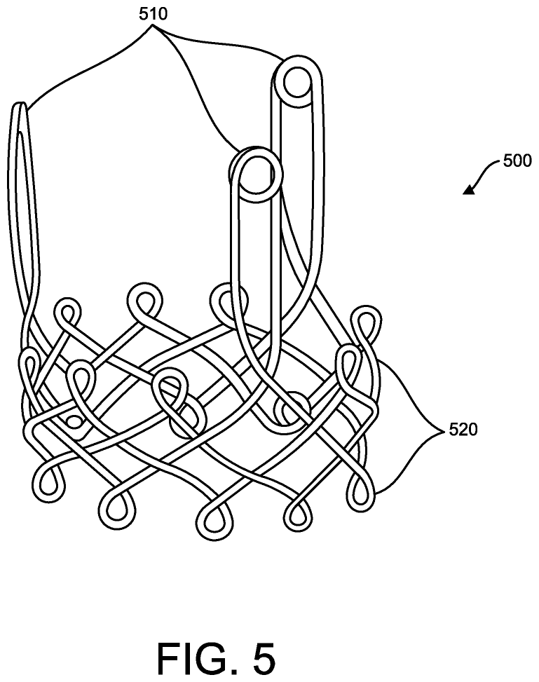

FIG. 5 generally illustrates an embodiment of a tubular, braided frame as disclosed herein. As shown in FIG. 5, a braided frame 500 may comprise commissure posts 510 and a sealing zone 520, wherein the braided frame 500 has a braided tube length that is shortened considerably when compared to other embodiments disclosed herein that still provides a minimal frame necessary for connection to leaflet structure.

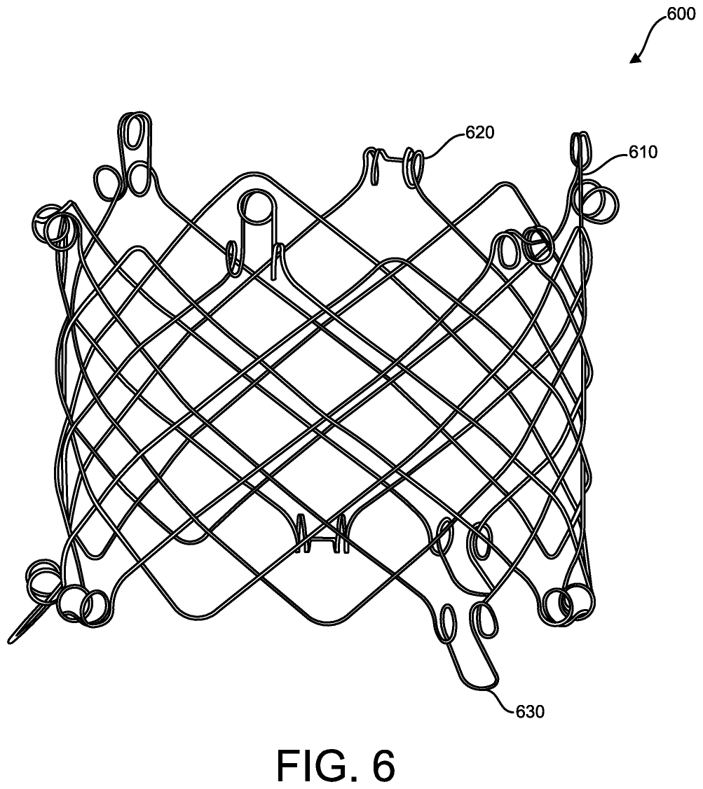

FIG. 6 generally illustrates an embodiment of a tubular, braided retrievable frame as disclosed herein. FIG. 6 discloses a braided frame 600 that comprises additional features, such as commissures 1610 with wire coils 620 and 630 that have an axis parallel to the tangent of the frame circumference. Coils 620 and 630 may act as springs to increase the strength of the commissures 610, which in turn provides resistance to flow forces during valve closure. Coils may be designed using parameters such as wire diameter, loop coil diameter, and coil turns to optimize valve performance. Further, coils 620 and 630 may comprise variations designed to create a latch for releasably joining the frame to a receiver. The round nature of the coils creates a spring-like latch for engaging a receiving geometry such as a cylinder or a custom, even asymmetrical, shape. The extended latch-feature of coil 630 may be used in concert with a leash, which would pull in on the latch coil 630, towards the center of the valve axis, bending the latch coil 630 to release from a receiver.

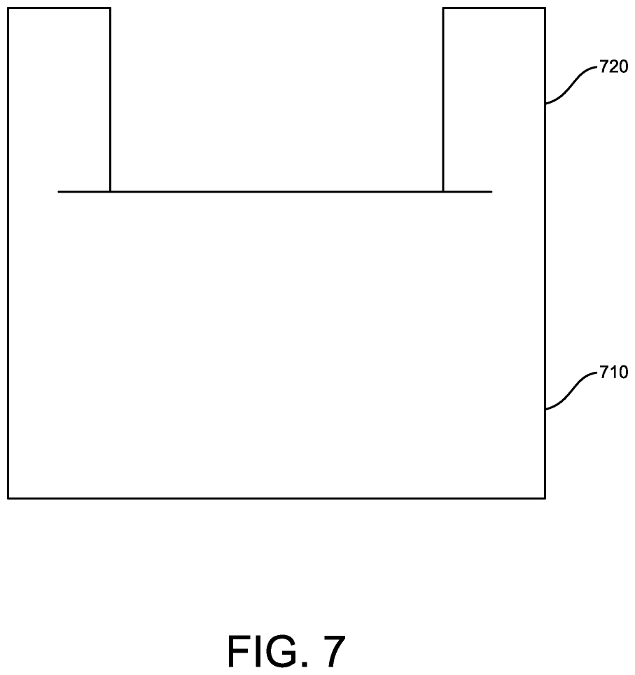

FIG. 7 generally illustrates an embodiment of a leaflet panel as disclosed herein. Further, FIG. 7 discloses a side view of a single leaflet pattern 710. In one embodiment, three leaflet patterns may be sewn, bonded, or otherwise connected to each other along the commissure seam 720. The leaflet can be made from animal tissue such as porcine or bovine pericardium or biocompatible fabric. The portions at the top, when sewn together, can be configured to form a pocket that fits over and adhere to a commissure post. In some embodiments, such as for a mitral replacement valve, the leaflet is from 0.005″ to 0.030″ thick and 1.5 cm to 3 cm along each side.

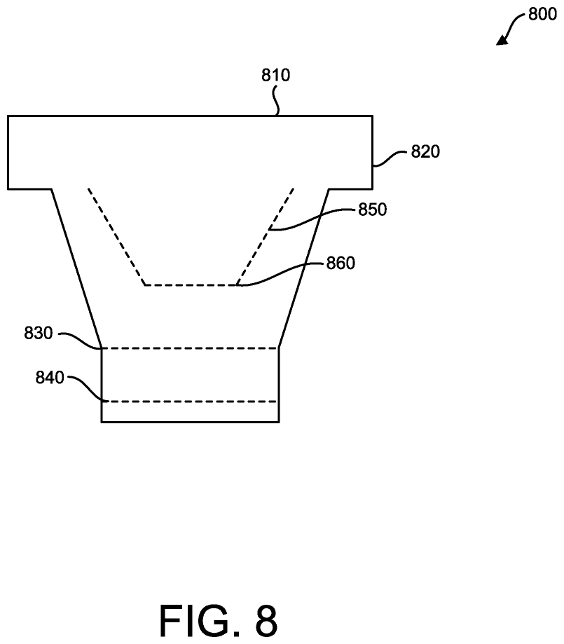

FIG. 8 generally illustrates an embodiment of a leaflet panel as disclosed herein. A leaflet panel may be defined as a pattern, cut from synthetic or biological material, that serves as a single leaflet. For example, excised porcine or bovine pericardium may be used for a leaflet panel. The combination and attachment of two or more leaflet panels create a leaflet assembly, such as a Z-valve insert. As shown in FIG. 8, a leaflet panel 800 may comprise a distal end 810 that when incorporated into a completed valve, becomes the co-apting closing zone of the valve, where the three proximal ends are forced together in a Y form, called a co-apt, to close the valve. The outer ends 820 of the distal end 810 have tabs that are used to sew the leaflets to the commissure posts. Towards the proximal end, a base stitch zone 830 is used to create a sealing zone, by itself or in combination with an inflow stitch zone 840. An inflow stitch 840 is generally parallel to and on the inflow side of the base stitch and serves to attach the Z-valve insert to a frame or cuff. The material between the inflow stitch and the base stitch can be a continuous part of the Z-valve insert, or be a different material sewn to the Z-valve insert.

A belly stitch is defined as a stitch originating at the edge seams of the Z-valve insert and following a wire to define an edge of a leaflet, with the option to attach to one of the wires of the frame and/or cuff. The wire where the belly stitch is attached may be shaped-set to further improve leaflet durability and performance. A cuff may be defined as additional material positioned either on the outside or inside of the frame and may be extended along the top and bottom of the frame, though at a minimum is attached above and below the base stitch. The belly stitch serves the purpose of improving leaflet durability and hemodynamic performance. A valve belly stitch 850 is angled from the distal outer sections towards the middle center of the leaflet and may, in one embodiment, either be sewn to the braided frame or an outer cuff. A bellows portion 860 creates the bottom of the belly and may or may not be sewn to the frame. A bellows portion 860 of the belly stitch 850 may be defined as an interruption of attachment or following of the belly stitch, generally at the center of the leaflet, that serves to improve collapsibility.

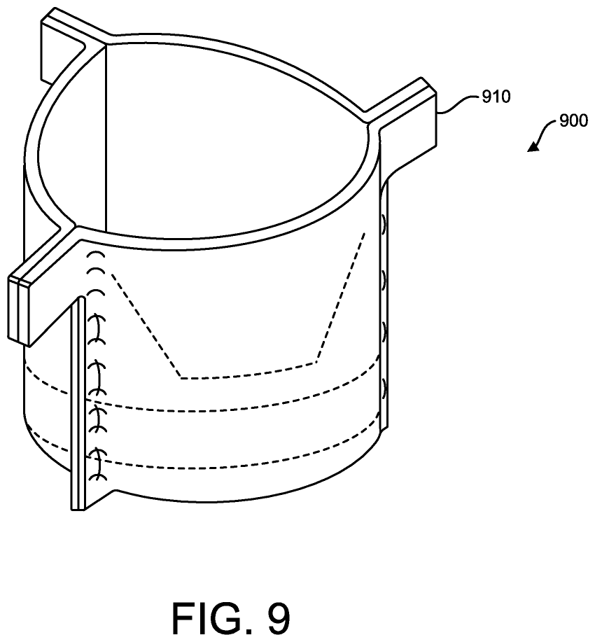

FIG. 9 generally illustrates an embodiment of a Z-valve insert as disclosed herein. As shown in FIG. 9, a Z-valve insert 900 may comprise three leaflet panels, with commissure ends 910 of each leaflet panel connected and stitched together along the ends to create a tube-like, cylindrical structure. The edges may be parallel or have a specified angle, so as to optimize durability and hemodynamic performance. A Z-valve insert 900 may also comprise an optional tubular portion on the inflow side of the base stitch, created by overlapping the lower tabs of the Z-valve insert 900. In one embodiment, the Z-valve insert 900 is attached to the commissure posts of a tubular frame via a base stitch. The base stitch may be located at the top, middle, or along the bottom of the tubular frame. In another embodiment, the base and inflow stitch lines may pass through the leaflets, the braid, and the cuff, wherein the region on the outside of the valve frame between the base stitch and the inflow stitch creates a sealing zone.

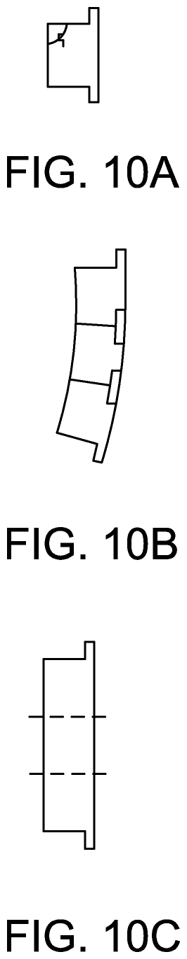

FIGS. 10A-10C generally illustrate embodiments of patterns for leaflet panels. The embodiment in FIG. 10A discloses a view of the leaflet assembly. FIG. 10B discloses a pattern combining three separate leaflets. FIG. 10C is a variation of the assembly showing a straight edge which allows for attachment of additional cuff material.

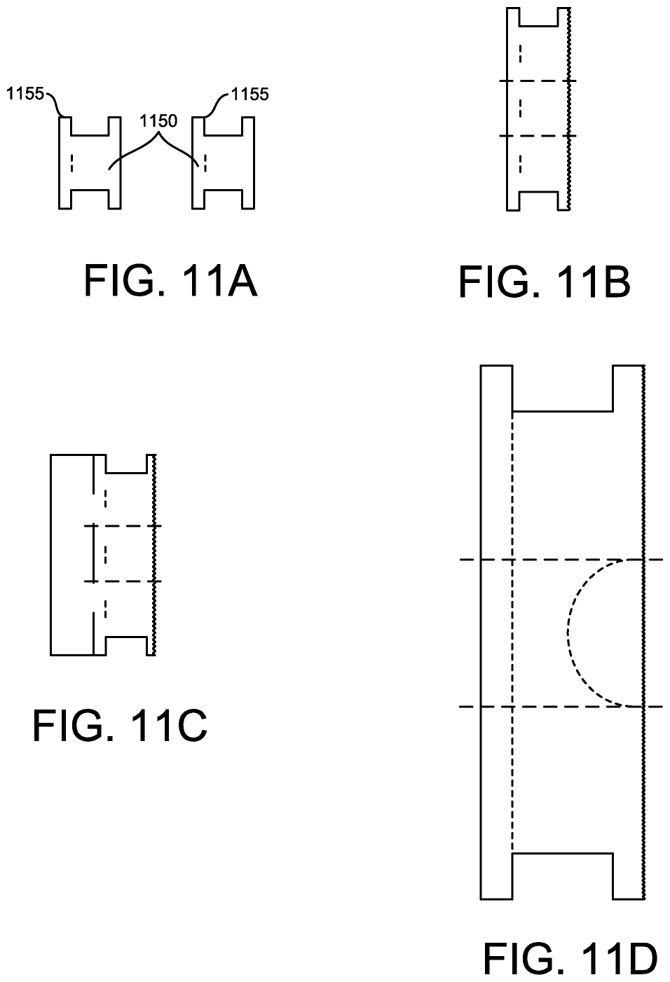

FIG. 11A discloses a preferred embodiment of a valve leaflet pattern 1150 with a cuff 1155 at the proximal end. The cuff 1155 may be used to wrap over the proximal end of a frame and create an outer sealing zone in addition to the inner sealing zone.

FIG. 11B shows three leaflets combined and incorporating a cuff.

FIG. 11C discloses a cuff of larger size, which can serve to seal the complete outer frame spanning the length of a valve assembly.

FIG. 11D discloses a scale version of a combined three-piece leaflet.

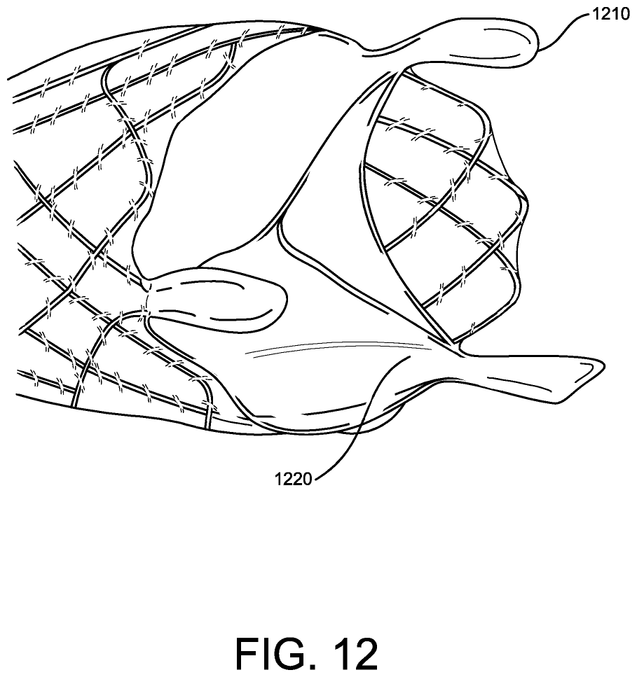

FIG. 12 generally illustrates an embodiment of a collapsible, heart-valve assembly system as disclosed herein. As shown in FIG. 12, the leaflets can be assembled to each other and/or to the frame by sewing. The leaflets can be joined at commissure seams 1210 and then sewn, welded or otherwise attached to the commissure posts as well as to other points on the frame such as wires or wire intersections, or to materials attached to the frame, for example the leaflets being attached to a cuff, which cuff is then attached to the frame. Once the assembly is complete, the leaflets work in concert to close on the outflow (distal side when being implanted into leaflets a mitral valve) when fluid pressure is increased distally, so that the leaflets close or co-apt work in a Y pattern 1220.

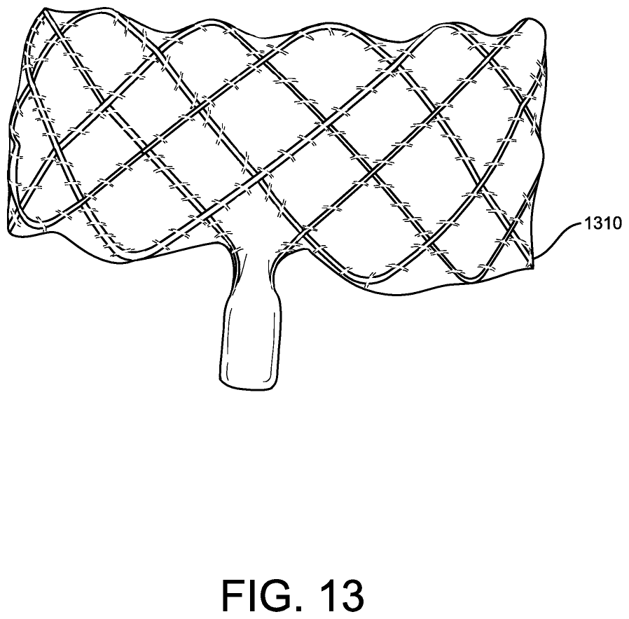

FIG. 13 generally illustrates an embodiment of a collapsible, heart-valve assembly system as disclosed herein. In one embodiment, a skirt 1310 conforms to and substantially covers the internal side of the frame, which skirt 1310 is also positioned between the leaflets and the retrievable frame portion. In the embodiment shown, the skirt 1310 increases in size to form a parachute-like feature pressuring the leaflets to co-apt and close when hydrostatic pressure is increased due to inflow of blood during diastole.

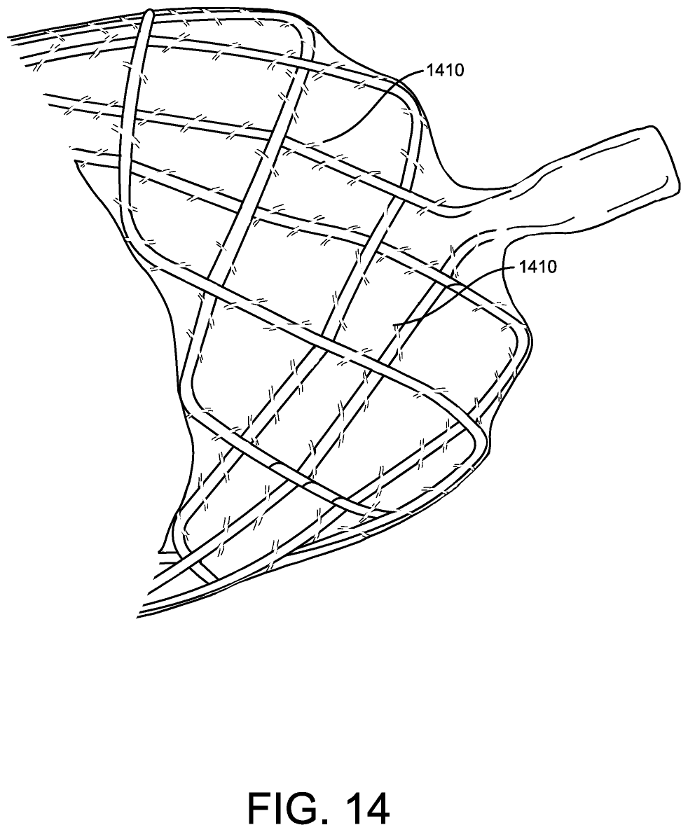

FIG. 14 generally illustrates an embodiment of a collapsible, heart-valve assembly system as disclosed herein. As shown in FIG. 14, the valve system may comprise sewing connections 1410 of the skirt and leaflets to each other and to the frame.

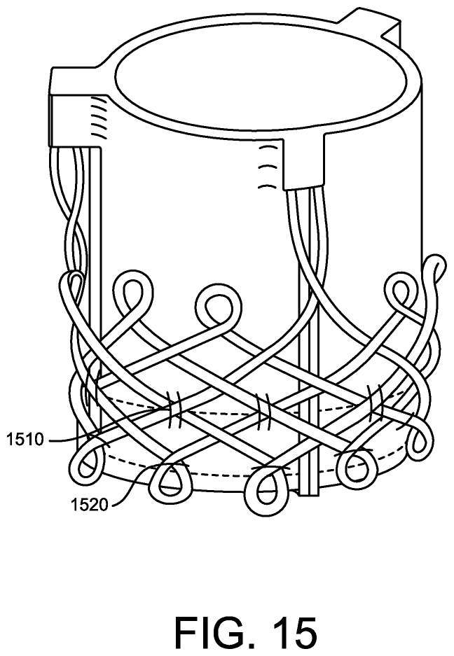

FIG. 15 generally illustrates an embodiment of a collapsible, heart-valve assembly system as disclosed herein. The valve assembly in FIG. 15 comprises an extended sealing zone created by a longer leaflet that is sewn to the structure at the centerline 1510 and baseline stitch, and also at the proximal braid end along inflow stitch line 1520.

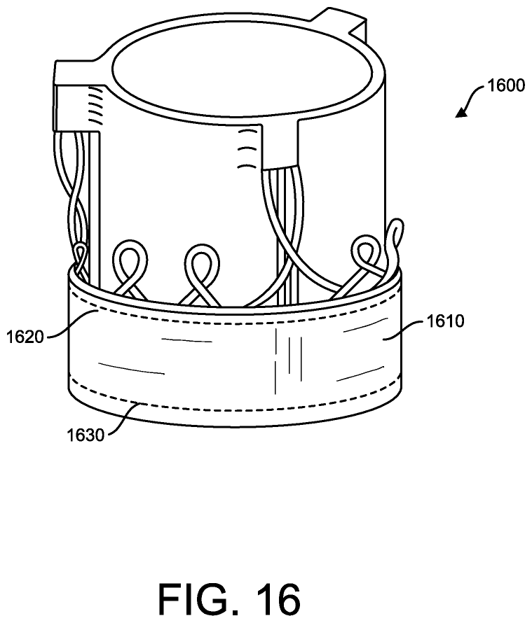

FIG. 16 generally illustrates an embodiment of a collapsible, heart-valve assembly system as disclosed herein. As shown in FIG. 16, the valve assembly 1600 may comprise—in addition to the tubular, braided frame and leaflet structure incorporated into the frame—a cuff 1610, wherein the cuff 1610 covers the proximal portion of the valve assembly 1600. A cuff 1610 may be defined as material that is attached to the frame, wherein the cuff 1610 may be positioned either on the outside or inside of the frame and may be extended along the top and bottom of the frame, and may be attached, in some embodiments, above and below the base stitch 1620. In another embodiment, the cuff 1610 may be attached alongside an inflow stitch line 1630. The cuff material may be: elastic and deform with the braid; non-elastic, wherein the braid wires slide through/around the attached cuff; or a combination of both. The cuff may also comprise a polymeric coating (e.g. chronosil) or a continuous knit, woven or braided fabric. And cuffs may be rolled up with a seam or a tubular structure.

In another embodiment, a continuous cuff may be sewn to the Z-valve insert at the baseline stitch location and wrapped around the inflow edge of the braided valve frame to become a cuff on the outer side of the frame. And in a separate embodiment, a valve may have both an inner cuff and an outer cuff, and/or partial cuffs that cover discrete portions of the braided valve frame.

Cuffs 1610 are generally used for covering the wires of the frame so as to provide a sealing zone, wherein a sealing zone, or ring, is formed to prevent blood flow from either side while the leaflets provide flow control. The sealing zone is comprised of either flexible or non-flexible material. Cuffs 1610 also serve the purpose of attaching the Z-valve insert to the frame. In a preferred embodiment, the cuff 1610 attached along the top and bottom edges of the frame, or along a row of crossing points. A cuff 1610 may be attached to the frame along all adjacent wires, such as with a stitch that does not interfere with the motion of the braid crossing points.

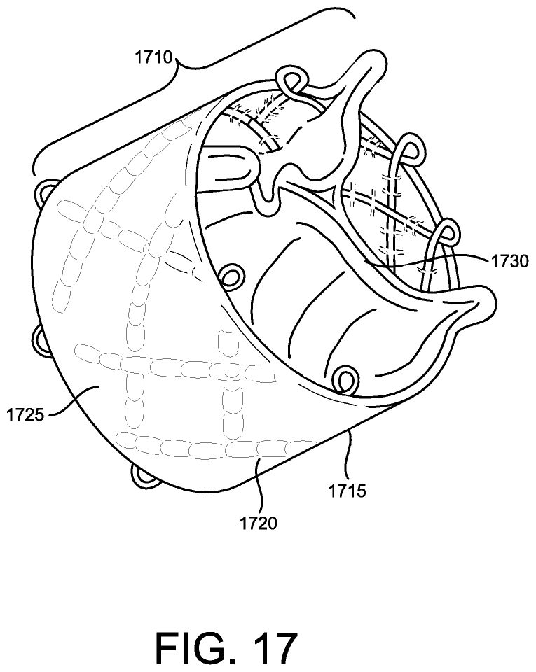

FIG. 17 generally illustrates an embodiment of a collapsible, heart-valve assembly system as disclosed herein. As shown in FIG. 17, in one embodiment, a valve assembly 1710 comprises a braided frame, and a long cuff covering 1715 on the outside. This cuff over the complete outer frame may serve as an extended sealing zone. A belly stitch 1720 may be sewn to the frame whereas a bellows stitch 1725 is not sewn to the frame. In this embodiment, the distal leaflet ends 1730 are shown co-apting so as to close the valve in a loose Y-shape. In some embodiments, the valve co-apt area may comprise some “looseness” so as to ensure sufficient and effective contact among all three leaflets and ensure complete closing of the valve. Leaflets may be constructed of tissue such as porcine pericardium or other materials known to the art. In some cases, valves or parts of valves excised from animals may be sewn into the disclosed frame structure.

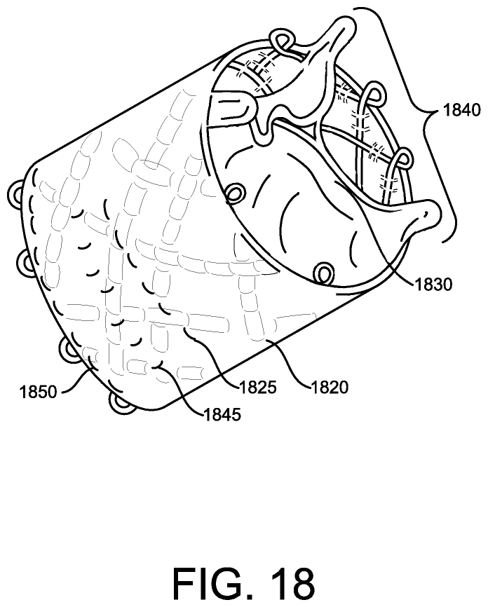

FIG. 18 generally illustrates an embodiment of a collapsible, heart-valve assembly system as disclosed herein. As shown in FIG. 18, a valve assembly may comprise combined commissure posts and leaftlet tabs 1840, a leaflet end, co-apt zone 1830, a belly stitch 1820, a bellows stitch 1825, a base stitch 1845, and an inflow stitch 1850.

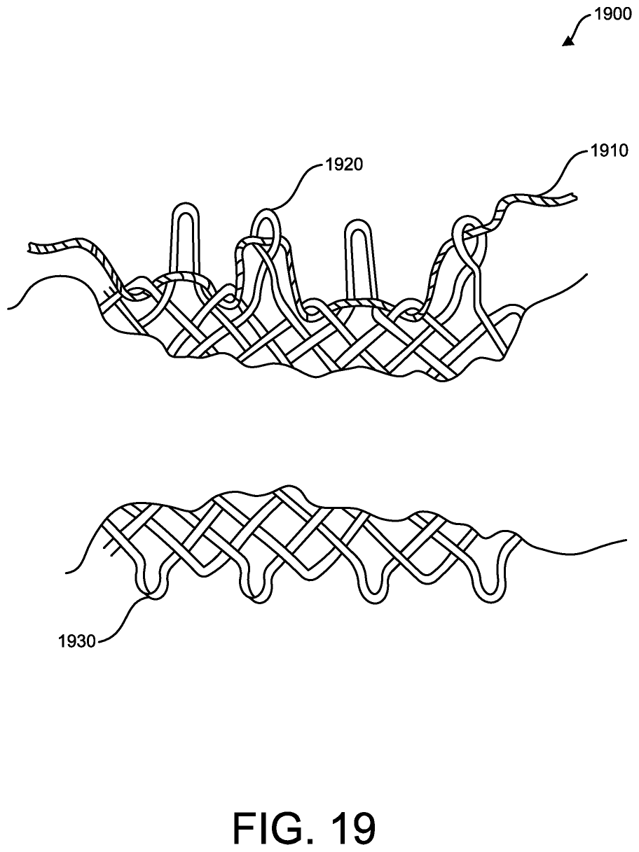

FIG. 19 generally illustrates an embodiment of a retrieval system of a collapsible, heart-valve assembly as disclosed herein. FIG. 19 discloses an embodiment of a retrieval system 1900 for the removal of a valve from the cardiac structure and receiver after delivery. The retrieval system 1900 may comprise a leash 1910, which may be permanently incorporated into the loops 1920 of the braided frame. In some embodiments, the leash 1910 may be comprised of radiopaque material for visibility under fluoroscopy. The leash 1910 may be captured by one or more retrieval hooks on a catheter that may then be pulled into the catheter or a specifically designed retriever. The tension of the leash 1910 and hooks may partially compress the valve, separating it from the receiver.

In one embodiment, the leash, also referred to as a capture string, is threaded in a complete circle around the inflow end of the retrievable frame. The leash 1910 may be threaded around commissure posts and through wire elbows. The leash 1910 can be used to retrieve the valve by the retrieval catheter. For example, the leash 1910 can be captured by a retrieval catheter and put in tension (i.e., pulled on) radially inwardly, which in turn pulls the frame inward radially and thus away from the permanently implanted receiver frame portion. Also shown in FIG. 19 are delivery catheter-connection elements 1930. In an embodiment, the delivery catheter-connection elements 1930 created at alternate elbows on the outflow end of the frame. In other embodiments, various parts of the valve system may be configured to vary in size, number and shape as desired; for example, to conform to an aortic valve or pulmonary valve, or to form-fit the parameters of a specific patient's heart and heart valve size, strength, configuration, disease condition, or otherwise as desired by a user.

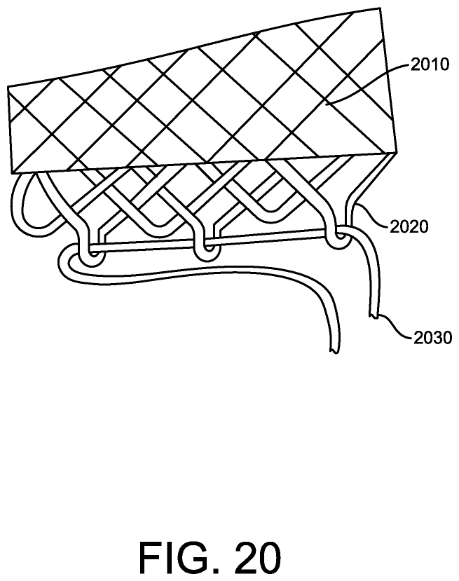

FIG. 20 generally illustrates an embodiment of a collapsible, heart-valve assembly system as disclosed herein. As shown in FIG. 20, delivery catheter-connection elements 2020 may be configured for engagement for delivery or retrieval, and for release from the delivery catheter by a lock suture or string 2030. Similar in the inverse to leash and its catheter attachment loops, delivery catheter-connection elements 2020 hold lock suture 2030. During delivery, the delivery catheter puts tension on (i.e., pulls on) the lock suture 2030 radially inwardly, which in turn pulls on the delivery catheter-connection elements 2020 inward radially, which holds the retrievable frame portion 2010 in a diametrically compact form, small enough to be held in the catheter and then freely within the capture elements of the permanently implanted receiver frame portion. The tension on the lock suture 2030 can then be released by the delivery catheter to cause the retrievable frame portion to expand radially and thereby to lock within the receiver frame portion. If needed, the tension on the lock suture 2030 can be repeatedly reapplied and released until the entire retrievable frame portion is placed exactly a desired position, orientation, etc., within the receiver frame portion.

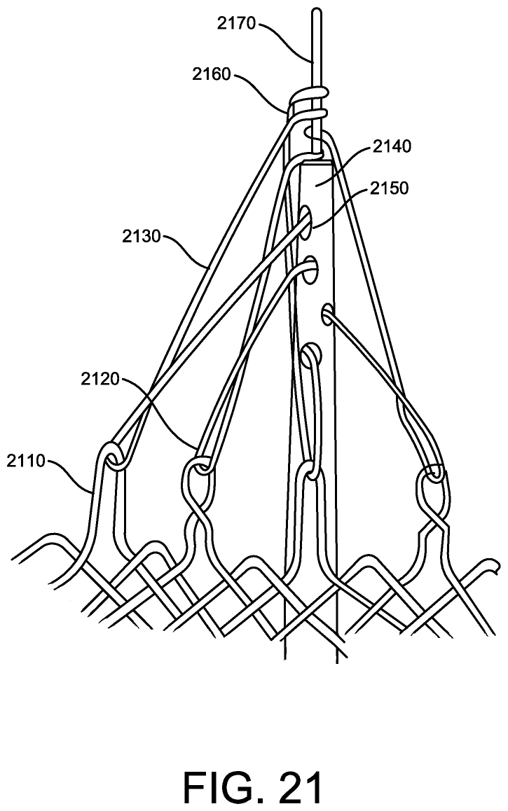

FIG. 21 generally illustrates an embodiment of a deployment system for a collapsible, heart-valve assembly system as disclosed herein. As shown in FIG. 21, the valve system may be inserted into a distal end of a delivery catheter. FIG. 21 further shows an example mechanism for delivery or retrieval. In this embodiment, a plurality of sutures 2130 are fixedly attached to a reel 2140 through ports 2150, and also slidably looped 2160 around a post or axle 2170 carried within the delivery catheter, for example in a coaxial arrangement. Each suture is also threaded through a commissure post 2110 or delivery feature 2120. The reel 2140 and axle 2170 are rotated by a drive mechanism to collapse or expand the retrievable frame portion. Once the retrievable frame portion is satisfactorily expanded and positioned, the axle 2170 is withdrawn relative to the reel 2140, releasing the distal loops 2160 so that the retrievable frame portion can expand fully, thus fully deploying the retrievable frame portion.

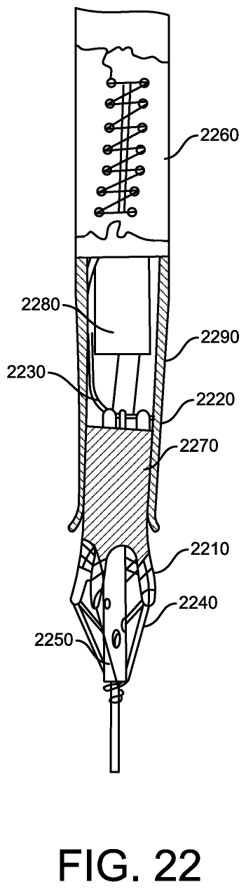

FIG. 22 generally illustrates an embodiment of a deployment system for a collapsible, heart-valve assembly system as disclosed herein. As shown in FIG. 22, the receiver frame valve assembly 2270 may include a delivery reel mechanism. The receiver frame valve assembly 2270, shown partially deployed in FIG. 22, is incased in a delivery catheter 2290, wherein the delivery catheter 2290 comprises a corset 2260. The receiver frame valve assembly 2270 is retained in collapsed form in the catheter by lock suture 2230, which engages delivery catheter-connection elements 2220 and is releasably locked to the delivery catheter. This lock suture 2230 can be used to keep the end of the valve within the delivery catheter 2290 until desired. A drive mechanism 2280 that engages the reel 2250 can be shown that would control the release of the distal valve.

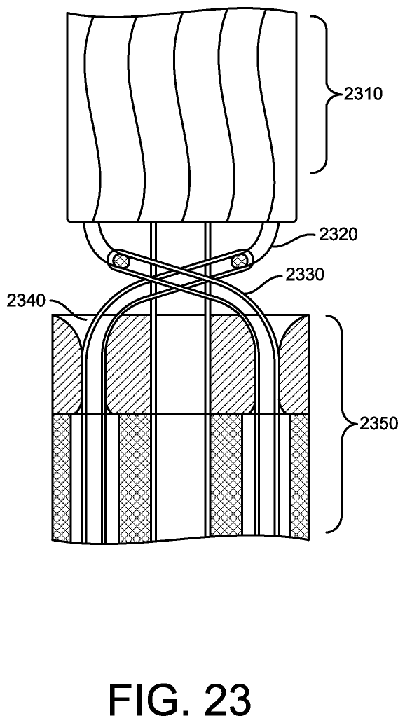

FIG. 23 generally illustrates an embodiment of a deployment system for a collapsible, heart-valve assembly system as disclosed herein. Preferably a proximal, or in-flow side, delivery system. As shown in FIG. 23, a delivery system may comprise a compressed valve 2310 and loops 2320 that are slidably retained by suture lines 2330. The suture lines 2330 follow a pathway through bushings 2340 incorporated into the delivery catheter 2350. The sutures 2330 may control the expansion and retention of the valve assembly as desired.

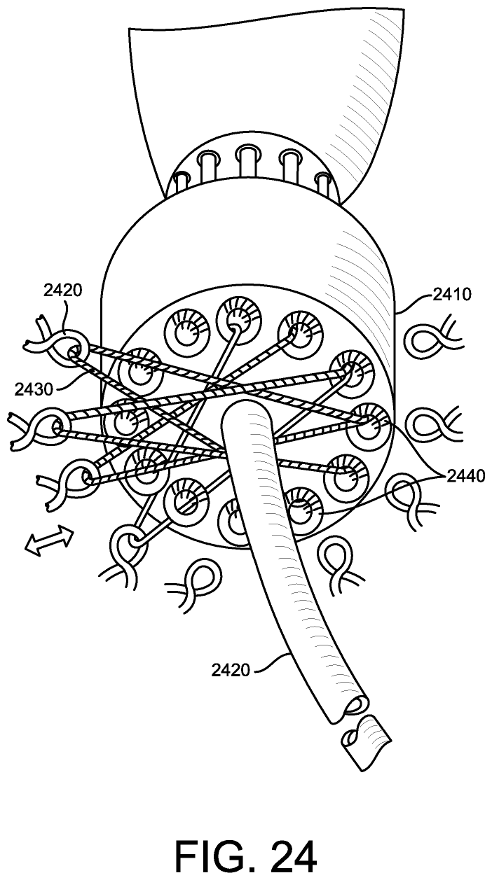

FIG. 24 generally illustrates an embodiment of a deployment system for a collapsible, heart-valve assembly system as disclosed herein. As shown in FIG. 24, each loop 2420 has a suture 2430 threaded through it, wherein the sutures 2430 exit from and return to bushings 2440 on a manifold 2410. The pattern of the sutures 2430 alternates in direction—starting from a bushing on the opposite side of a loop, through the loop, and then back to a bushing, such that the sutures create tension with a vector through the centerline. This has the benefit of providing the aforementioned centerline tension vector for each frame loop, while avoiding the tubing 2420 that extends through the center of the delivery catheter. This pattern also has the benefit of providing tension to draw down on and keep the loops as close to the center as possible. As one skilled in the art may appreciate, each suture has an end that is fixed relative to the delivery catheter, and an opposite end which, when properly configured in length, can be pulled or released in concert with the rest of the sutures to precisely control the expansion of the valve. When the valve is to be released, the sutures can be cut/released and withdrawn. This mechanism can be used to preferentially control the expansion of the proximal valve' but could be used to control the distal valve as well.

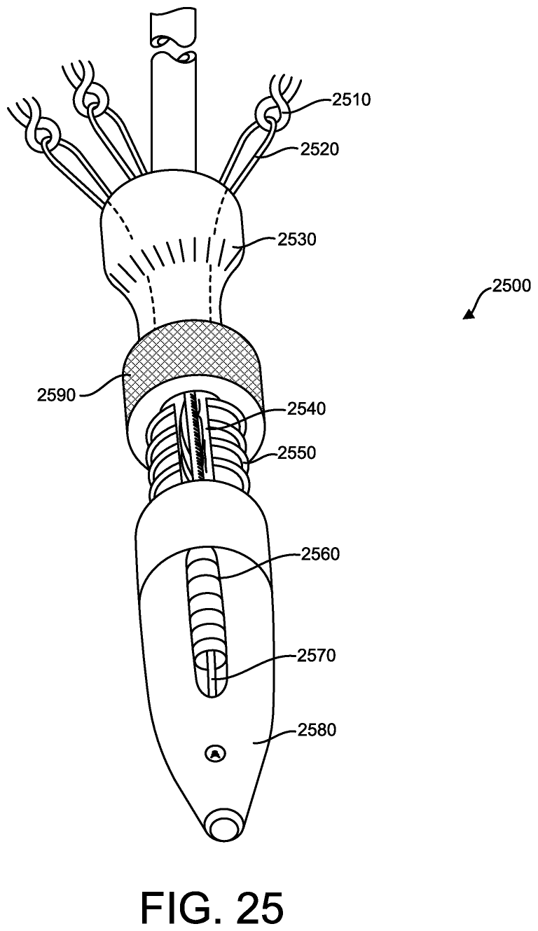

FIG. 25 generally illustrates an embodiment of a deployment system for a collapsible, heart-valve assembly system as disclosed herein. As shown in FIG. 25, a delivery system 2500 for control of a distal valve may comprise distal valve loops 2510 that are slidably connected to short-length sutures 2520. The sutures 2520 are guided through a funnel bushing 2530, through a channel 2540 in a threaded rod 2550, and terminated with loops 2560 on a wire 2570, which is slidably retained in distal tip 2580. The wire 2570 is accessible at the proximal end of the delivery system. A knob 2590 is turned to push the bushing 2530 prior to delivery to pull the loops 2510 to a compressed position. The valve is released by pulling the wire 2570, releasing one end of the sutures 2520.

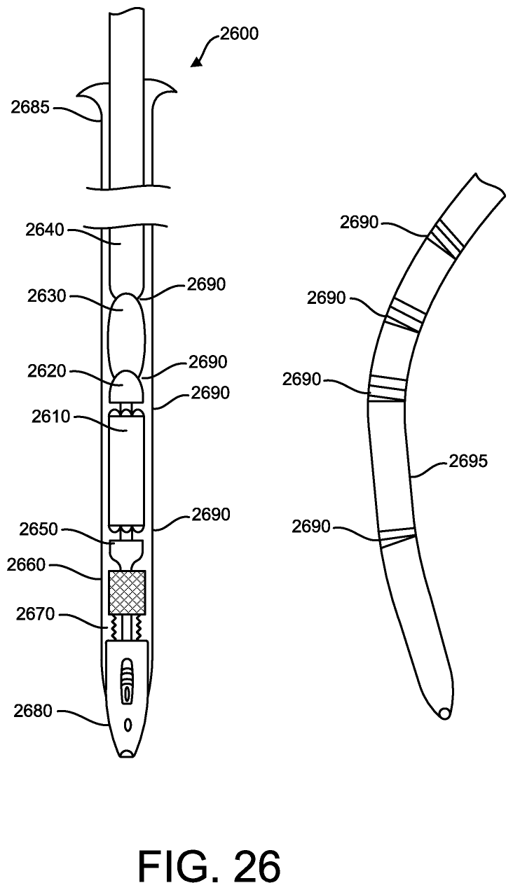

FIG. 26 generally illustrates an embodiment of a deployment system for a collapsible, heart-valve assembly system as disclosed herein. FIG. 26 discloses a distal cut-out, side view 2600 and a distal outside-view 2695 of combined assemblies along with additional structures. FIG. 26 discloses a valve 2610 in a compressed form, with a proximal release-mechanism manifold 2620. The manifold 2620 may be abutted to a bearing 2630, that allows some angular articulation between it and the manifold 2620 and possibly another bushing 2640. These bushings can provide some angular articulation while allowing the sutures and center tubing through them. The release mechanism for the distal valve comprises a funnel bushing 2650, a knob 2660, a threaded rod 2670, and a distal tip 2680. Also shown is an outer sheath 2685, which is pushed over the assembly prior to delivery to ensure complete compression of the valve and smooth outer surface for insertion in the body. This sheath may then be retracted proximally from outside the body to expose the valve and release mechanisms. Angular articulation zones are shown at points 2690, where some amount of flexibility is gained by the structure design.

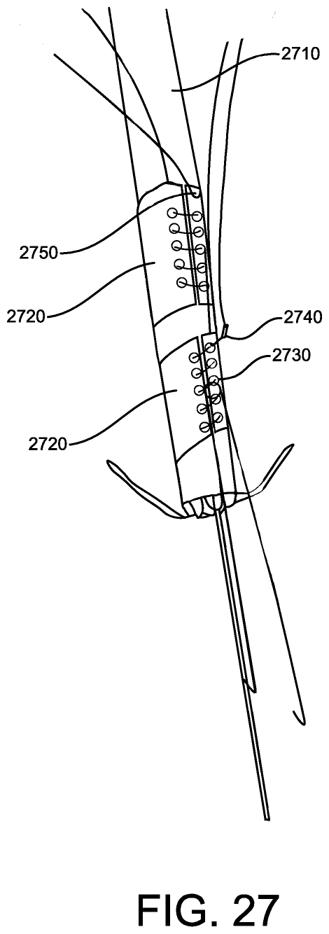

FIG. 27 generally illustrates an embodiment of a deployment system for a collapsible, heart-valve assembly system as disclosed herein. As shown in FIG. 27, a deployment system, such as a delivery catheter 2710, may comprise one or more corsets 2720 around it and positioned directly over or in close proximity to the valve system compressed within the deployment system. Corsets 2720 address challenges incurred during transcatheter placement of the valve system. Specifically, during transcatheter delivery of the valve system, the deployment system must make a sharp turn of approximately 180 degrees over a short distance of a few centimeters to deliver the valve system to the native valve. Because the compressed valve system exerts significant force against sheaths within the deployment system, the sharp turn inhibits release of the self-expanding valve system: pulling back of the sheath is difficult or impossible and attempts to pull it back around a sharp turn result in displacement of the catheter and valve. Placement of the corset 2720 on the deployment system minimizes displacement of the catheter and valve system.

In a preferred embodiment, a corset 2720 is rectangle- or square-shaped and made of a non-compliant fabric, such as dacron, ripstop nylon, or similar fabrics. The fabric of the corset 2720 comprises a strong, tight weave, non-compliant in both x and y directions, with a thickness that may range 0.002-0.006″ (inches) and corresponding weights. The fabric of the corset 2720 may be formed into a tube, such that the meeting ends of the fabric are in close proximity but not overlapping. The meeting ends may have alternating stitches 2730—termed, releasing sutures—that loop around a pull wire 2740 and back in a manner that, when in place, the pull wire 2740 keeps the meeting ends together, and when the pull wire 2740 is pulled out through the releasing sutures, the meeting ends are released and separate. In some embodiments, the pull wire 2740 may be configured to attach to a portion of the corset 2720 for retrieval after the wire is pulled though the releasing sutures. Pull wires may comprise bent ends to prevent accidental removal during assembly. Upon fmal assembly, the bends at the ends of the pull wires are snipped off.

Pull wires 2740 may be constructed of very flexible stainless steel or nitinol, typically 0.010-0.020″ in diameter, and coated with friction-reducing coatings such as teflon, paralene, silicone, or the like. The pull wires 2740 may be translated from the distal end of the catheter where the valve assembly is, to the proximal end of the delivery catheter that employs a handle for controlling the catheter and securing/releasing the pull wires 2740 and retrieval leashes 2750. The catheter may employ a single lumen through which all the wires and leashes translate, it may have multiple lumens for each wire and leash, and it may comprise combinations thereof. Where the delivery system employs a larger guide catheter that the delivery system translates through, the released corsets can be pulled into this guide using the leashes and subsequently removed. The combination of the corset and pull wires provide the benefit over prior art of a release system wherein the forces to pull the wires are very small and can be done easily around a 180-degree turn, and so displacement of the catheter and valve is minimized.

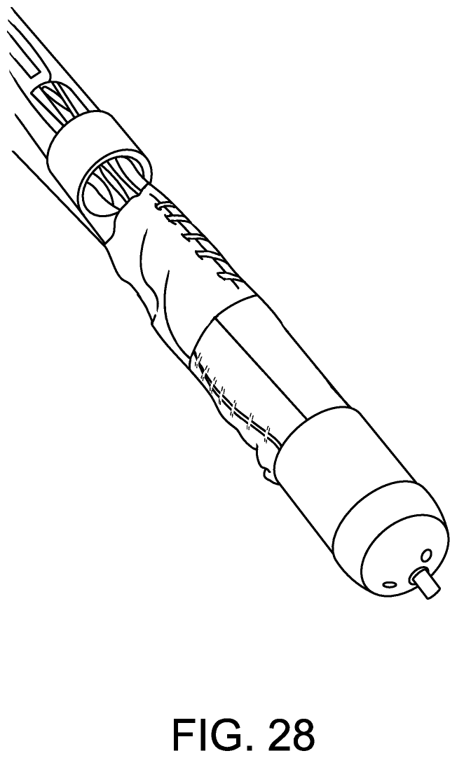

FIG. 28 generally illustrates an embodiment of a deployment system for a collapsible, heart-valve assembly system as disclosed herein.

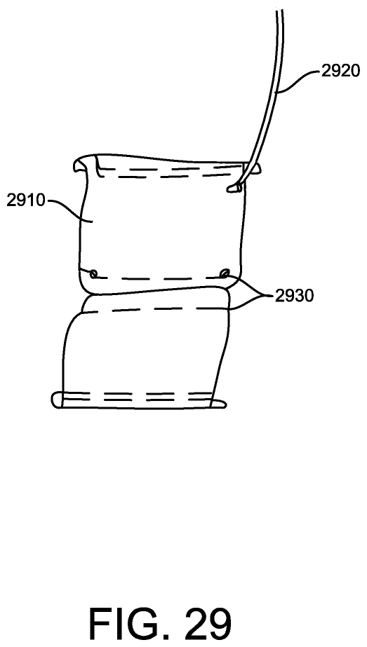

FIG. 29 generally illustrates an embodiment of a corset of a deployment system for a collapsible, heart-valve assembly system as disclosed herein. As shown in FIG. 29, a corset 2910 may comprise an attached leash 2920 for the purpose of retrieving the corset 2910 after release, wherein the leash is made from a wire, string, or suture. FIG. 29 further discloses releasing stitches 2930 of the corset 2910. In one embodiment, a corset is formed over a 0.375″ OD, 0.350″ID tube (27F). The valve assembly is compressed using a stent crimping tool and loaded inside the tube. The corset and valve assembly are then simultaneously pushed out of the tube such that the corset is then over the valve assembly.

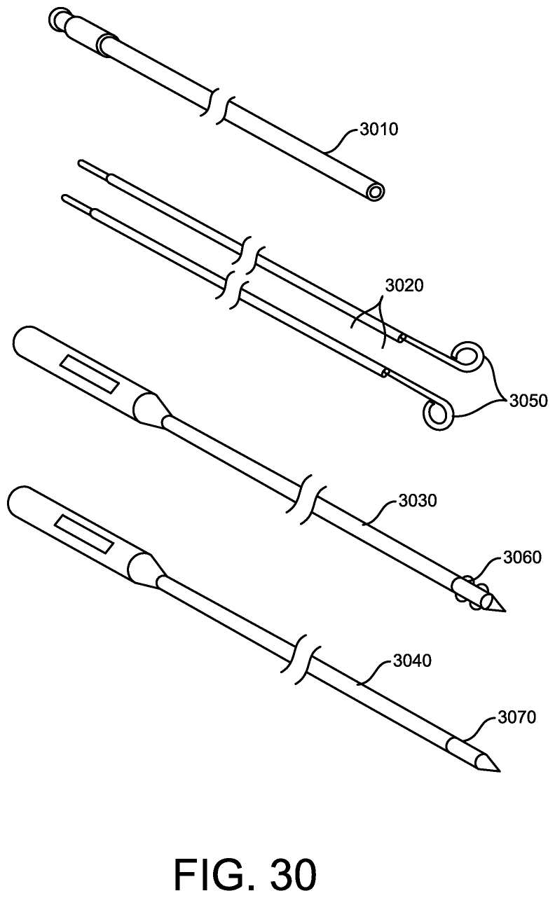

FIG. 30 generally illustrates an embodiment of a deployment system for a collapsible, heart-valve assembly system as disclosed herein. As shown in FIG. 30, a guiding catheter 3010 may be used to deploy the valve assembly, such as a commercially available delivery catheter or a custom catheter, or one acting as a simple conduit for delivery of other system components. In one embodiment, two rails 3020 may extend from the catheter (more or fewer rails can be used if desired) to deliver anchors 3050 to the chordae tendineae. A receiver delivery catheter 3030 may deliver the receiver frame portion 3060. And a valve-and-retrievable-frame assembly catheter 3040 may deliver the valve and retrievable frame assembly 3070.

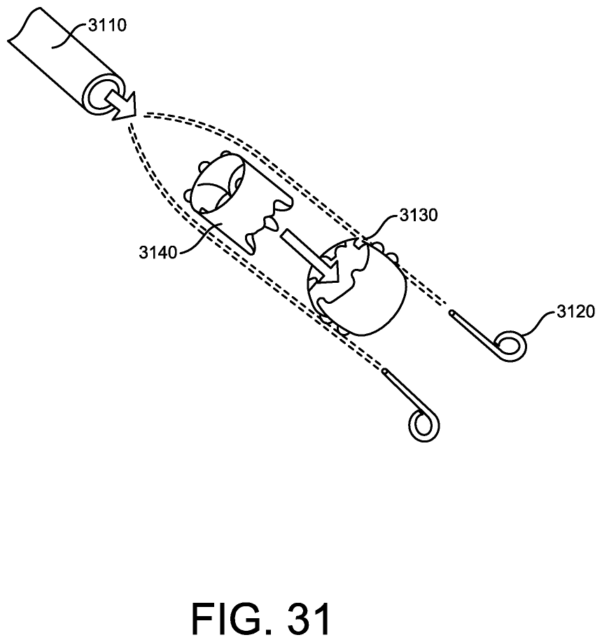

FIG. 31 generally illustrates an embodiment of a deployment system for a collapsible, heart-valve assembly system as disclosed herein. As shown in FIG. 31, a replacement heart-valve system may comprise a valve and retrievable frame assembly 3140 (comprising a tissue valve or other blood-flow regulator held within a retrievable frame portion) sized and configured to releasably connect to a cooperatively configured receiver frame portion 3130. In one embodiment, the receiver frame portion 3130 comprises an anchor system 3120, which may anchor into the chordae tendineae of a valve, such as a mitral valve. This is transmitted to the target site in the native heart via guiding catheter 3110, and the components can be delivered in sequence and/or in an already connected forma, with the anchors 3120 placed first into the target site via the rails (shown as dotted lines). In another embodiment, the receiver frame portion 3130 is sized and configured to releasably connect to a cooperatively configured anchor system 3120, which may anchor into the chordae tendineae of a valve, such that when delivered, the receiver frame portion 3130 is placed into and attached to the anchors 3120, after which the valve and retrievable frame assembly 3140 is placed inside the receiver frame portion 3130.

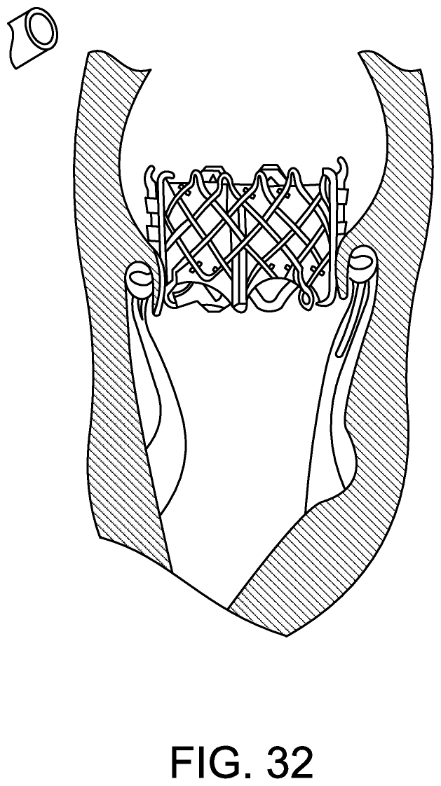

FIG. 32 generally illustrates an embodiment of a collapsible, heart-valve assembly system as disclosed herein. As shown in FIG. 32, the anchors of a receiver frame portion may be locked in place in a native target heart valve, wherein the receiver frame portion has the capacity to hold the heart-valve system.

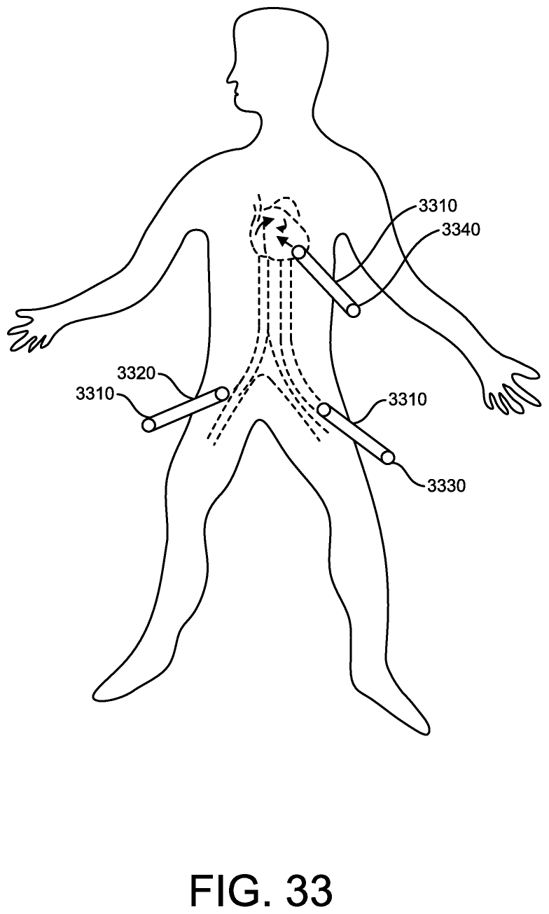

FIG. 33 generally illustrates an embodiment of a deployment system for a collapsible, heart-valve assembly system as disclosed herein. As shown in FIG. 33, several methods are available for deployment of the components of the replacement heart valve system to a desired target cardiac structure. FIG. 33 depicts at least three different pathways suitable to deliver the components to a mitral valve structure. A “transapical” approach comprises inserting the guiding catheter 3310 in the groin 3320 into a vein and up to the mitral valve via the atrial septum. A “transaortic” approach comprises inserting the guiding catheter 3310 into the groin 3330 into an artery and then up to the mitral valve via the aortic valve. An alternative “transapical” approach 3340 comprises surgically exposing the heart and inserting the guiding catheter 3310 into the apex of the target heart.

In other embodiments, a guiding catheter may be placed in the patient's body first, then the rails, which can be separate from or releasably attached to receiver frame portion either in the guiding catheter or in the target site, are placed through the catheter into the target site. The anchors are engaged to desired cardiac structures such as the chordae tendineae. Next, the receiver frame portion is placed through the guiding catheter after the proximal ends of the delivery guiding rails are threaded through collars on the receiver frame portion, the receiver frame portion attached to the anchors and thus placed at the desired target native cardiac structure location. The receiver frame portion is then released from the receiver delivery guiding catheter, which catheter then is withdrawn from at least the heart, and typically the body, of the patient. The anchors are then released from the rails, and the rails are withdrawn. Subsequently, a valve delivery catheter is placed in the guiding catheter and the valve and retrievable frame assembly is delivered to and connected to the receiver frame portion, for example by inserting valve and retrievable frame assembly partially or completely into the receiver frame portion and/or mechanically attaching the valve and retrievable frame assembly to the receiver frame portion.

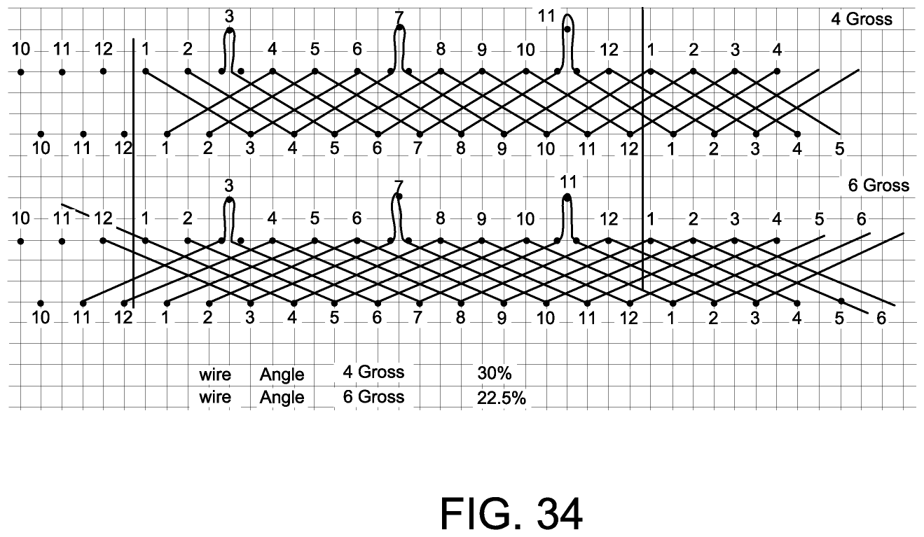

FIG. 34 generally illustrates an embodiment of a braiding configuration for the retrievable frame as disclosed herein. FIG. 31 discloses two exemplary braiding configurations for the retrievable frame portion. The configurations disclose an embodiment where the wires cross 4× per circumference and an embodiment where the wires cross 6× per circumference. The embodiments also show commissure posts and identify exemplary angles at wire intersections.

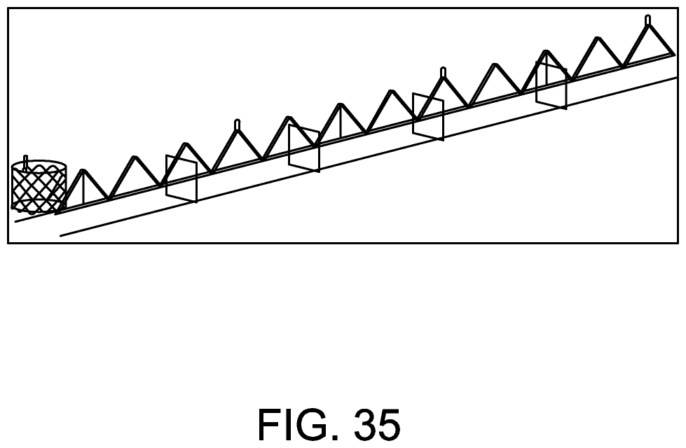

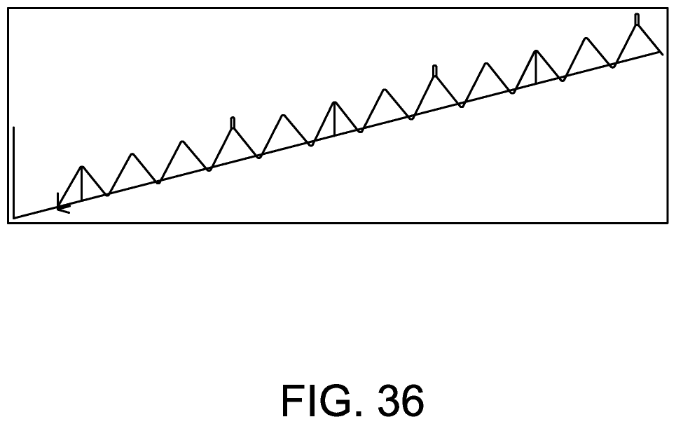

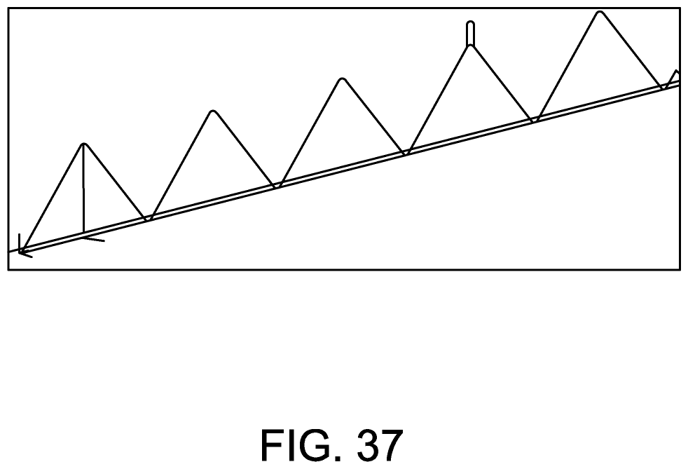

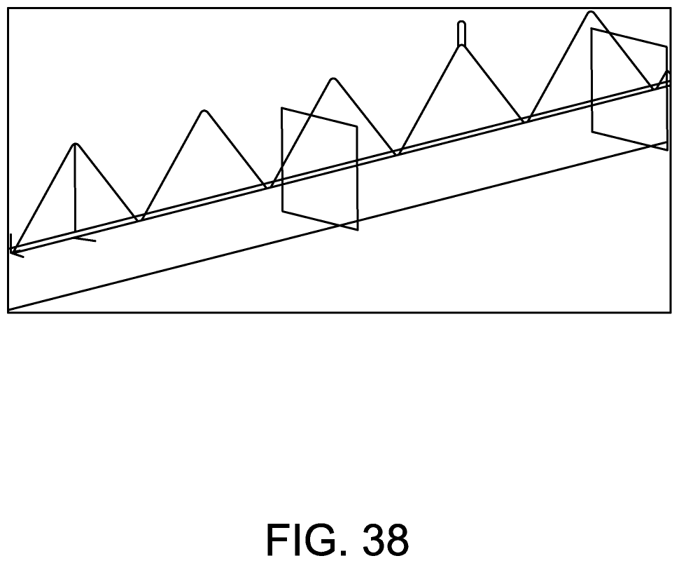

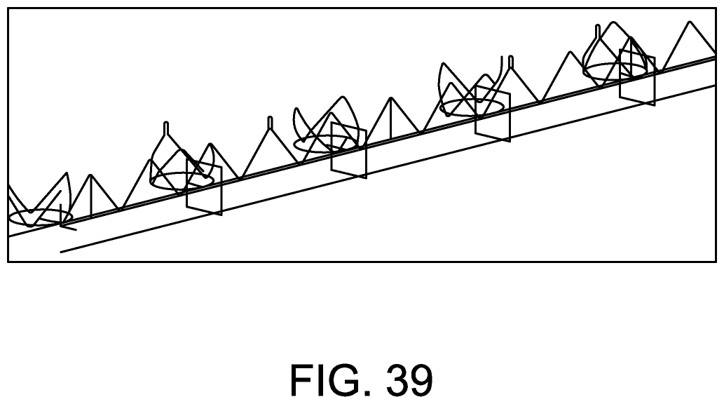

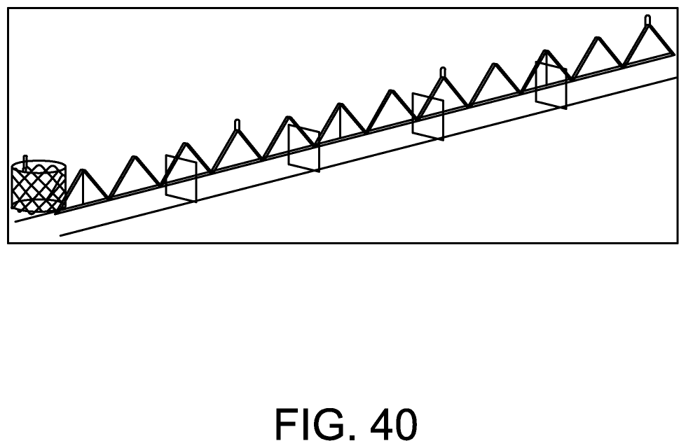

FIG. 35-40 generally illustrates an embodiment of a braiding configuration for the retrievable frame as disclosed herein.

In other embodiments, the replacement heart valve systems can also be implemented and/or used as follows: The rails and anchors are translated percutaneously across the target heart valve. A sheath carrying the rails and to implement anchors through the guiding catheter is pulled back partially to expose the hook of the anchor, and the hook is then manipulated to engage the valve chordae and/or other sub-valvular structure (depending in part on the target valve). Once the hooks are satisfactorily engaged in the target site, the receiver frame portion, which is configured to hold a retrievable frame portion, is loaded onto the rails and slid into place such that the mating features on the receiver frame portion are properly located on corresponding mating zones of the anchor. The receiver frame portion is then expanded. Once expanded, the receiver and anchors combine to be essentially one complete anchor/receiver unit, i.e., an in-situ anchor-receiver frame unit. The anchors are then released from the delivery guiding catheter by disengaging the wire lock from the torque tube and pulling proximally. The valve and retrievable frame assembly is then delivered through the same or a different catheter and attached in situ to the anchor/receiver unit.

The embodiments depicted above are not limiting. Other delivery mechanisms are possible, including for example combining the receiver frame portion and the valve and retrievable frame assembly into one delivery catheter, combining the rail/anchors and the receiver frame portion into one delivery catheter, or various combinations of the above.

In other embodiments, frame-variations comprise a suitable apex-bend radii at the time of manufacture (for when the frame is being constructed on a mandrel) of a retrievable frame portion include an internal radius of 0.2 mm, 0.3 mm, 0.4 mm, or 0.5 mm. Such radii can be reduced to 0.1 mm or less when the frame is collapsed to in-catheter configurations.

Suitable materials to construct the frames, leashes, coatings, etc., herein can be selected and configured as desired or needed by a user or patient. For example, material coverings or coatings can be selected and configured to elicit specific cellular and molecular responses, such as endotheliazation promotors that promote endothelial coverage of the frame, anchors. Other coatings, etc., can be selected to enhance biocompatibility but inhibit or eliminate tissue ingrowth/integration. This endotheliazation inhibition can enhance the ability of a device herein to be removed from a target site while breaking minimal adhesions, i.e., cellular connections, between the frames herein or between the frames and the target tissue. Examples of pro-endotheliazation agents include), and pro-VEGF factors. Examples of anti-endotheliazation agents include medically acceptable fucans and anti-VEGF drugs such as bevacizumab (monoclonal antibody); ranibuzumab (antibody derivative); pegaptanib (aptamer); lapatinib, sunitinib, and sorafenib (oral small molecule agents that inhibit tyrosine kinases); VEGF trap-eye (fusion proteins), and miscellaneous agents such as siRNA-bevasiranib and adPEDF.

In some embodiments, pro-bioactivity and anti-same-bioactivity agents are provided on different, selected areas of the devices, systems, etc., herein, including for example with a single frame. For example, a pro-endothelization agent can be provided in one location and an anti-endothelization agent provided in another location. The agents' locations can be configured, for example, to enhance in-growth of permanent elements such as anchors or connection native-tissue elements of receiver frames (if any) while enhancing free movement or easy removal of non-native-tissue-connection elements. Native tissue responses can also be affected by a user's choice of material or coating properties including but not limited to chemistry/composition, structural dimension, pore size, and surface topography.

In other embodiments, the materials of the valve system can be inert or can have a controlled or predictable reactivity with or to surrounding native tissue. Reactivity mechanisms can be selected to elicit cellular reactions such as promoting endothelial attachment of the native target site to permanently implanted portions of the replacement heart valve system, thereby promoting biocompatibility. In some embodiments, reactivity mechanisms of the frame material and/or its coatings can be selected to elicit moderate-to-aggressive tissue ingrowth integrating the material into the native tissue such as native myocardium. In some embodiments, reactivity mechanisms of the frame material and/or its coatings can be selected to suppress specific reactions such as thrombosis or inflammation/scarring. One example of such an anti-scarring/anti-fibrous adhesion material is medically acceptable fucan.

Coatings and other materials can also be selected to promote biocompatibility. Suitable coatings include polymers and chemical vapor deposited ceramics such as low temperature isotropic carbon. Coatings and other materials can include drugs having a selected, specific therapeutic effect. Fabric-type materials can be a fabric of woven, braided, knit, etc. Materials can also form a film or sheet, such as a polymer film, that spans between supporting members, or struts. One example of a suitable material is ePTFE, including ePTFE having a selected, desired porosity.

Physical characteristics of the frame can include a time-dependent response such as one frame portion of the device deploying to final configuration more slowly relative to another frame portion of the device to ensure proper sequence of actions. Such time-dependent response can be varied between the retrievable frame portion and receiver frame portion, or within different sections of a single frame.

Materials in and/or between the frame(s) of the delivery system can also be selected and configured to assist in deployment/retrieval of the device. One example of such a configuration is to place material between deployment or control wires extending from the catheter to create an “umbrella” that modulates blood flow during delivery. Materials such as fabrics can be advantageously used in such configurations, or to provide physical support or restraint of frame members relative to one another, or to attach leaflets.

Additional desirable characteristics can include selected lubricity of the components, for example by providing a Teflon material, so that lubricity between the frames, or the system herein against the interior of the delivery catheter(s), is increased, and thus friction decreased, to reduce the force to deploy the device. This can also improve tactical feedback to the surgeon or other user.

Properties of the materials, etc., herein, including for example coatings, fabric, and tissue, may be varied in localized areas of the device including within the valve leaflets, within a frame or between one frame and the next. For example, a material can be configured elicit a reaction of tissue ingrowth that diminishes from one part of the structure relative to another—such as moving inward radially. Similarly, coatings can be removed or selectively deposited to vary the characteristics of a localized area of the device.

Other embodiments may include combinations and sub-combinations of features described or shown in the several figures, including for example, embodiments that are equivalent to providing or applying a feature in a different order than in a described embodiment, extracting an individual feature from one embodiment and inserting such feature into another embodiment; removing one or more features from an embodiment; or both removing one or more features from an embodiment and adding one or more features extracted from one or more other embodiments, while providing the advantages of the features incorporated in such combinations and sub-combinations. As used in this paragraph, “feature” or “features” can refer to structures and/or functions of an apparatus, article of manufacture or system, and/or the steps, acts, or modalities of a method.

References throughout this specification to “one embodiment,” “an embodiment,” “an example embodiment,” etc., indicate that the embodiment described may include a particular feature, structure, or characteristic, but every embodiment may not necessarily include that particular feature, structure, or characteristic. Moreover, such phrases are not necessarily referring to the same embodiment. Further, when a particular feature, structure, or characteristic is described in connection with one embodiment, it will be within the knowledge of one skilled in the art to affect such feature, structure, or characteristic in connection with other embodiments whether or not explicitly described.

Unless the context clearly indicates otherwise (1) the word “and” indicates the conjunctive; (2) the word “or” indicates the disjunctive; (3) when the article is phrased in the disjunctive, followed by the words “or both,” both the conjunctive and disjunctive are intended; and (4) the word “and” or “or” between the last two items in a series applies to the entire series.

Where a group is expressed using the term “one or more” followed by a plural noun, any further use of that noun to refer to one or more members of the group shall indicate both the singular and the plural form of the noun. For example, a group expressed as having “one or more members” followed by a reference to “the members” of the group shall mean “the member” if there is only one member of the group.

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

What is claimed is:

1. A replacement heart-valve system comprising:
a tubular braided frame, wherein the tubular braided frame comprises an inflow end and an outflow end;
a leaflet assembly, wherein the leaflet assembly comprises at least one valve leaflet, wherein the at least one leaflet comprises an inflow end, an outflow end, and one or more commissure tabs extending horizontally away from the inflow end;
wherein the one or more commissure tabs of the at least one leaflet is connected to the inflow end of the tubular braided frame.

2. The replacement heart-valve system of claim 1, wherein the tubular braided frame further comprises one or more commissure posts extending vertically away from the inflow end, wherein the one or more commissure tabs of the at least one leaflet is connected to the one or more commissure posts of the tubular braided frame.

3. The replacement heart-valve system of claim 1, wherein the tubular braided frame further comprises one or more looping structures on one or both of the inflow end and the outflow end.

4. The replacement heart-valve system of claim 2, wherein the tubular braided frame further comprises one or more looping structures within the one or more commissure posts.

5. The replacement heart-valve system of claim 1, wherein the tubular braided frame further comprises one or more wire coils extending horizontally away from one or both of the inflow end and outflow end.

6. The replacement heart-valve system of claim 1, wherein the tubular braided frame is a braid of one or more wires;
wherein the braid of the one or more wires is either a zig-zag braid or an over-under braid; and
wherein the one or more wires consists of one of: nitinol wire, stainless steel, cobalt chrome, and nylon.

7. The replacement heart-valve system of claim 1, further comprising a cuff along one or both of the inflow end and the outflow end of the tubular braided frame, wherein the cuff is positioned either on the inside or the outside of the tubular braided frame.

8. The replacement heart-valve system of claim 1, further comprising a skirt along one or both of the inflow end and the outflow end of the tubular braided frame.

9. The replacement heart-valve system of claim 1, wherein the leaflet assembly comprises two or more valve leaflets, wherein the one or more commissure tabs of the two or more leaflets are connected and wherein the outflow end of the two or more valve leaflets are connected to form a y-shape.

10. The replacement heart-valve system of claim 9, wherein the connected one or more commissure tabs and the connected outflow end of the two or more valve leaflets are connected by one or more of: sewing, fusing, and stitching.

11. The replacement heart-valve system of claim 1, wherein the at least one valve leaflet is made of one or both of: an animal tissue and a polymer.

12. The replacement heart-valve system of claim 1, wherein one or more rows of stitching connects the leaflet assembly to the tubular braided frame, wherein the one or more rows of stitching is circumferentially positioned in accordance with one or more of the following: along the inflow end of the tubular braided frame, along the outflow end of the tubular braided frame, and between the inflow end and outflow end of the tubular braided frame.

13. A method of preparing a replacement heart-valve system, comprising:
a tubular braided frame, wherein the tubular braided frame comprises an inflow end, an outflow end; and
a leaflet assembly, wherein the leaflet assembly comprises at least one valve leaflet, wherein the at least one leaflet comprises an inflow end, an outflow end, and one or more commissure tabs extending horizontally away from the inflow end; and
connecting the one or more commissure tabs of the at least one leaflet to the inflow end of the tubular braided frame.

14. The method of claim 13, wherein the tubular braided frame further comprises one or more commissure posts extending vertically away from the inflow end, and wherein the method further comprises connecting the one or more commissure tabs of the at least one leaflet to the one or more commissure posts of the tubular braided frame.

15. The method of claim 13, wherein the tubular braided frame further comprises one or more looping structures on one or both of the inflow end and the outflow end.

16. The method of claim 14, wherein the tubular braided frame further comprises one or more looping structures within the one or more commissure posts.

17. The method of claim 13, wherein the tubular braided frame further comprises one or more wire coils extending horizontally away from one or both of the inflow end and outflow end.

18. The method of claim 13, wherein the tubular braided frame is a braid of one or more wires;
wherein the braid of the one or more wires is either a zig-zag braid or an over-under braid; and
wherein the one or more wires consists of one of: nitinol wire, stainless steel, cobalt chrome, and nylon.

19. The method of claim 13, further comprising connecting a cuff along one or both of the inflow end and the outflow end of the tubular braided frame, wherein the cuff is connected either on the inside or the outside of the tubular braided frame.

20. The method of claim 13, further comprising connecting a skirt along one or both of the inflow end and the outflow end of the tubular braided frame.
